(19) United States
(12) Patent Application Publication
(10) Pub. No.: US 2008/0200531 A1
(43) Pub. Date: Aug. 21, 2008

(54) CDKI pathway inhibitors as inhibitors of tumor cell growth

(76) Inventors: Bey-Dih Chang, Madison, WI (US); Igor B. Roninson, Loudonville, NY (US); Donald Porter, Middle Grove, NY (US)

Correspondence Address: KEOWN & ZUCCHERO, LLP, 500 WEST CUMMINGS PARK, SUITE 1200, WOBURN, MA, 01801, US

(21) Appl. No.: 11/803,739

(22) Filed: May 15, 2007

Related U.S. Application Data
(60) Provisional application No. 60/747,220, filed on May 15, 2006.
(60) Provisional application No. 60/849,968, filed on Oct. 6, 2006.

(51) Int. Cl. A61K 31/40; C12Q 1/48

(52) U.S. Cl. 514/416; 435/15

(57) ABSTRACT

The invention provides new methods for inhibiting the CDKI pathway and specifically inhibiting tumor cell growth. The invention further provides new and specific inhibitors of tumor cell growth, as well as means for discovery of additional such inhibitors. The present inventors have surprisingly discovered that Cyclin-Dependent Kinase 3 (CDK3) is specifically required for tumor cell growth, in contrast to other members of the CDK family.

### RELATED APPLICATIONS

This application claims the benefit of U.S. Provisional Application Ser. No. 60/747,220, filed May 15, 2006 and U.S. Provisional Application Ser. No. 60/849,968, filed Oct. 6, 2006. The entire teachings of the above-referenced Application are incorporated herein by reference.

### BACKGROUND OF THE INVENTION

1. Field of the Invention

The invention relates to the inhibition of tumor cell growth. More particularly, the invention relates to the inhibition of tumor cell growth through inhibition of the CDKI pathway.

2. Summary of the Related Art

Programmed cell cycle arrest occurs in a variety of physiological situations, such as damage response, growth factor depletion, contact inhibition, terminal differentiation of postmitotic cells, and senescence. Vidal and Koff, Gene 247: 1-15 (2000), teaches that all of these situations involve transient and/or permanent upregulation of cyclin-dependent kinase inhibitor (CDKI) proteins. CDKIs induce cell cycle arrest by inhibiting cyclin/cyclin-dependent kinase (CDK) complexes, which mediate transitions between different phases of the cell cycle. CDKI proteins belong to Cip/Kip or Ink4 protein families. Roninson, Cancer Letters 179: 1-14 (2002), teaches that the most pleiotropic of these proteins is p21^{Waf1/Cip1/Sdi1}, which inhibits different cyclin/CDK complexes and plays a role in damage-induced checkpoint arrest, induction of senescence and terminal differentiation. Sharpless, Experimental Gerontology 39: 1751-1759 (2004) teaches that among the other CDKIs, the CDK4/6 inhibitor p16^{Ink4a}, a tumor suppressor frequently inactivated in different cancers, has been implicated as the principal regulator of the maintenance of cell cycle arrest in senescent cells, whereas Vidal and Koff, supra, teaches that CDK2 inhibitor p27^{Kip1} is a key mediator of contact inhibition. Roninson, supra; Blagosklonny, Cell Cycle 1: 391-393 (2002); Blain et al., Cancer Cell 3: 111-115 (2003); and Weiss et al., Cancer Letters 189: 39-48 (2003), teach that tumor expression of several CDKI proteins, including p21, p27 and even p16, showed dual, both positive and negative, correlations with patient prognosis in several types of cancer. Martin-Caballero et al., Cancer Research 61: 6234-6238 (2001), teaches that, in animal studies, p21-null mice showed a higher frequency of spontaneous cancers but at the same time were resistant to radiation-induced carcinogenesis. Most strikingly, Blain et al., supra, and Weiss et al., supra, teach that inhibitors of p21 or p27 were found to inhibit tumor growth or drug resistance, and these CDKI were proposed as promising new targets for cancer therapeutics.

The surprising oncogenic associations of CDKI proteins have been explained through several types of experimental findings. LaBaer et al., Genes Dev. 11: 847-862 (1997), teaches that CDKI proteins act not only as inhibitors of cyclin/CDK complexes but also as potentiators of their assembly. Dotto, Biochimica et Biophysica Acta 1471: M43-M56 (2000), teaches that p21^{WAF1/Cip1} acts as an inhibitor of caspases and other apoptotic factors. Denicourt and Dowdy, Genes Dev. 18: 851-855 (2004), teaches that Cip/Kip proteins act as inhibitors of the invasion-suppressing Rho pathway, an activity specifically associated with cytoplasmic p27 or p21.

The most general pro-carcinogenic effect of CDKI proteins has emerged, however, from studies previously conducted by some of the instant inventors. This effect is transcriptional stimulation of multiple genes encoding different classes of secreted mitogenic, anti-apoptotic, angiogenic and pro-invasive factors, which results in paracrine tumor-promoting activity of CDKI-arrested cells. This insight came principally from cDNA microarray analysis of the effects of p21, which was expressed in a human fibrosarcoma cell line from an inducible promoter, as described in Chang et al., Proc. Natl. Acad. Sci. USA 97: 4291-4296 (2000). This analysis showed that p21 produces significant changes in the expression of multiple genes. A large number of genes are strongly and rapidly inhibited by p21, and most of these genes are involved in cell proliferation, with the single largest category functioning in the process of mitosis. Zhu et al., Cell Cycle 1: 59-66 (2002), teaches that inhibition of cell cycle progression genes by p21 is mediated by negative cis-regulatory elements in the promoters of these genes, such as CDE/CHR. Chang et al., Proc. Natl. Acad. Sci. USA 99: 389-394 (2002), teaches that the same genes are downregulated in tumor cells that undergo senescence after chemotherapeutic treatment, but p21 knockout prevents the inhibition of these genes in drug-treated cells. Hence, p21 is responsible for the inhibition of multiple cell cycle progression genes in response to DNA damage.

Chang et al., 2000, supra, teaches that another general effect of p21 induction is upregulation of genes, many of which encode transmembrane proteins, secreted proteins and extracellular matrix (ECM) components. Chang et al., 2002, supra, teaches that this effect of p21 is relatively slow, occurring subsequently to growth arrest and concurrently with the development of the morphological features of senescence. These genes are induced by DNA damage but p21 knockout decreases their induction. This decrease is only partial, which can be explained by recent findings that the majority of p21-inducible genes are also induced in response to other CDKI, p16 and p27 (see WO 03/073062). Gregory et al., Cell Cycle 1: 343-350 (2002) and Poole et al., Cell Cycle 3: 931-940 (2004), have reproduced gene upregulation by CDKI using promoter constructs of many different CDKI-inducible genes, indicating that it occurs at the level of transcription. Perkins et al., Science 275: 523-526 (1997); Gregory et al., supra and Poole et al., supra, teach that induction of transcription by p21 is mediated in part by transcription factor NFκB and transcription cofactors of the p300/CBP family. Unfortunately, other intermediates in the signal transduction pathway that leads to the activation of transcription in response to CDKI, the CDKI pathway, remain presently unknown.

Chang et al., 2000, supra, discusses medical significance of the CDKI pathway as indicated by the known functions of CDKI-inducible genes. Many CDKI-upregulated genes are associated with cell senescence and organism aging, including a group of genes implicated in age-related diseases and lifespan restriction. Migliaccio et al., Nature 402: 309-313 (1999), teaches that knockout of p66^{Shc}, a mediator of oxidative stress, expands the lifespan of mice by about 30%. Other CDKI-induced genes play a role in several age-related diseases, including Alzheimer's disease, amyloidosis, atherosclerosis, arthritis, renal disease and viral diseases. Merched and Chan, Circulation 110: 3830-3841 (2004), teaches that p21-null mice are resistant to experimental induction of atherosclerosis. Al-Douahji et al., Kidney International 56: 1691-1699 (1999) and Megyesi et al., Proc. Natl. Acad. Sci. USA 96: 10830-10835 (1999) teach that p21-null mice are resistant to experimental induction of chronic renal disease.

The strongest associations for CDKI-inducible genes, however, have been found in cancer. In particular, p21 expression activates the genes for many growth factors, inhibitors of apoptosis, angiogenic factors, and invasion-promoting proteases. In accordance with these changes in gene expression, Chang et al., 2000, supra teaches that p21-arrested tumor and normal cells show paracrine mitogenic and anti-apoptotic activities. Krtolica et al., Proc. Natl. Acad. Sci. USA 98: 12072-12077 (2001) and Parrinello et al., J. Cell Science 118: 485-496 (2005), demonstrated paracrine tumor-promoting activities in vitro and in vivo, respectively, in CDKI-expressing normal senescent fibroblasts, which express p21 and p16. Importantly, senescent fibroblasts possess the characteristic pro-carcinogenic activity that has long been identified with tumor-associated stromal fibroblasts. Roninson, 2002, supra, teaches that all of the experimental treatments shown to endow fibroblasts with tumor-promoting paracrine activities also induce the CDKI, suggesting that the CDKI pathway could be the key mediator of the pro-carcinogenic activity of stromal fibroblasts. Castro et al., The Prostate 55: 30-38 (2003); Michaloglou et al., Nature 436: 720-724 (2005) and Collado et al., Nature 436: 642 (2005), teach that the CDKI pathway is also activated in various pre-malignant conditions characterized by the senescent phenotype. te Poele et al., Cancer Res. 62: 1876-1883 (2002) and Roberson et al., Cancer Res. 65: 2795-2803 (2005), teach that the CDKI pathway is also activated in tumors that frequently become senescent as a result of chemotherapy. Stein et al., Cancer Res. 64: 2805-2816 (2004), in a recent bioinformatics study, identified 13 genes, expression of which is associated with the most intractable cancers.

CDKI proteins interact with different members of the CDK family. Various CDKs have been identified, including CDK1, CDK2, CDK3, CDK5 and CDK4/CDK6. Previous cancer-related studies have focused on CDK1, CDK2 and CDK4/CDK6. CDK1, CDK2 and CDK4/CDK6 (the latter two CDKs are closely related to each other and interact with the same class of cyclins), have been used as targets for developing specific inhibitors, with potential anticancer activity. Among the known inhibitors of CDKs, some commercially available selective inhibitors of CDK1 (CGP74514A) (Calbiochem Cat. No. 217696), CDK2 (CVT-313) (Calbiochem Cat. No. 238803), and CDK4 (NSC 625987) (Calbiochem Cat. No. 219477) have been developed and tested, as has been a broad-specificity CDK inhibitor, flavopiridol.

In contrast, CDK3 has not been used as a target for developing selective inhibitors. Meyerson et al., EMBO J. 11, 2909-2917 (1992) reports that CDK3 was discovered in the early 1990s, along with other related members of the CDK protein family. Human CDK3 protein comprises 305 amino acids; it shares 76% amino acid identity with CDK2 and 67% identity with CDK1/CDC2. Hofmann and Livingston, Genes Dev. 10, 851-861 (1996) teaches that CDK3 protein binds to E2F1 transcription factor, which is involved in G1/S transition. van den Heuvel and Harlow, Science 262, 2050-2054 (1993) teaches that a dominant-negative mutant of CDK3 induces G1 cell cycle arrest in mammalian cells. Meikrantz and Schlegel, J. Biol. Chem. 271, 10205-10209 (1996) teaches that such a dominant-negative mutant suppresses apoptosis. On the other hand, Park et al., J. Neurosci. 17, 8975-8983 (1997) teaches that a CDK3 mutant failed to inhibit apoptosis of neural cells, and Braun et al., Cell Biol. 17, 789-798 (1998) teaches that overexpression of CDK3 sensitizes mammalian cells to Myc-induced apoptosis. Interestingly, Braun et al., Oncogene 17, 2259-2269 (1998) teaches that CDK3 had no oncogenic activity and did not enhance c-Myc's transformation potential of rat embryo fibroblasts, but that high levels of CDK3 (but not CDK2) enhanced Myc-induced proliferation and anchorage-independent growth in Rat-1 cells. More recently, Ren and Rollins, Cell 117, 239-251 (2004) teaches that the complex of CDK3 with Cyclin C mediates the exit of mammalian cells from G0 state (quiescence) into G1 stage of the cell cycle.

Despite these reports, there have been strikingly fewer studies on CDK3 than on other CDK proteins. Among the possible reasons for this surprising lack of CDK3 studies may be that the most commonly used laboratory strains of mice do not express CDK3 due to a germline mutation in this gene (Ye et al., Proc. Natl. Acad. Sci. U.S.A 98, 1682-1686 (2001)), and that CDK3 is expressed at exceedingly low levels in all human tissues. Perhaps in part as a result of this, CDK3 has not been pursued as a potential target for anticancer drugs and no selective inhibitors of CDK3 have been previously developed.

There is a need for inhibitors of the CDKI pathway. There is also a need for novel CDK inhibitors with selectivity for cancer cells.

### BRIEF SUMMARY OF THE INVENTION

The present inventors have identified several compounds with the following CDK-related activity. These compounds inhibit the CDKI pathway, defined as the induction of transcription of multiple genes in response to the expression of a CDK inhibitor protein, such as p21^{Waf1}. These CDKI pathway inhibitors, designated SNX9 class compounds, also have a desirable ability to inhibit the growth of different types of tumor cells preferentially to normal cells.

The invention provides new methods for specifically inhibiting tumor cell growth. The invention further provides new and specific inhibitors of tumor cell growth, as well as means for discovery of additional such inhibitors. The present inventors have surprisingly discovered that Cyclin-Dependent Kinase 3 (CDK3) is specifically required for tumor cell growth, in contrast to other members of the CDK family.

In a first aspect, the invention provides a method for selectively inhibiting tumor cell growth comprising selectively inhibiting in a tumor cell cyclin-dependent kinase 3 (CDK3).

In a second aspect, the invention provides a method for identifying a specific inhibitor of tumor cell growth, the method comprising contacting an in vitro complex of a purified cyclin and CDK3 under conditions in which the complex of purified cyclin and CDK3 is capable of exhibiting kinase activity with a candidate inhibitor of such activity, and measuring the kinase activity of such complex in the presence or absence of such candidate compound.

In a third aspect, the method provides specific inhibitor compounds of CDK3, including compounds identified by the method according to the second aspect of the invention.

In a fourth aspect, the invention provides a method for treating a patient having a tumor with compounds that inhibit the induction of transcription by cyclin-dependent kinase inhibitors. The method according to this aspect of the invention comprises administering to a patient having a tumor a compound according to the invention.

In a fifth aspect, the invention provides a method for inhibiting the Cyclin-Dependent Kinase Inhibitor (CDKI) pathway downstream of the CDKI proteins and upstream of genes that are transcriptionally activated by the CDKI pathway. This method may have a variety of clinical applications in chemoprevention and therapy of different age-related diseases.

As a practical measure of the method according to this aspect of the invention, the method should not inhibit the essential tumor-suppressive role of CDKI proteins, nor should it directly inhibit the function of proteins encoded by genes that are transcriptionally activated by the CDKI pathway. However, inhibition of transcription of genes that are transcriptionally activated by the CDKI pathway is not regarded as direct inhibition of the function of proteins encoded by genes that are transcriptionally activated by the CDKI pathway.

### BRIEF DESCRIPTION OF THE DRAWINGS

FIG. 1 shows the effects of SNX9 on p21—induced CMV-GFP expression. Left bars show normalized GFP expression without p21 induction. Right bars show normalized GFP expression after three days of p21 induction.

FIG. 2 shows reversal of p21-induced transcription of firefly luciferase from the NK4 promoter by SNX9 relative to an unrelated compound.

FIG. 3 shows Q-PCR analysis of the effects of SNX9 and SNX9-1 on the induction of CDKI-responsive endogenous genes by p21. Left bars show results of carrier with no compound. Middle bars show results of 10 μM SNX9. Right bars show results of 20 μM SNX9-1.

FIG. 4 shows Q-PCR analysis of the effects of SNX9 on the induction of CDKI-responsive endogenous genes by p16. Left bars show results of carrier with no compound. Right bars show results of 10 μM SNX9.

FIG. 5 shows that SNX9 does not inhibit binding of NF-κB proteins p50 or p65 to double-stranded DNA oligonucleotide comprising NF-κB binding site. Each set shows oligonucleotide binding to p50 in control cells (left bars) and in cells treated with known NF-κB inducer TNFα (second bars), as well as oligonucleotide binding to p65 in control (third bars) or TNFα-treated cells (right bars). The left set of bars represents cells treated with carrier control, the middle set represents cells treated with SNX9, and the right set represents cells treated with a known inhibitor of NF-κB binding (TPCK).

FIG. 6 shows cell cycle effects of SNX9, as determined by FACS analysis of DNA content.

FIG. 7 shows results of DNA content and mitotic staining of untreated or SNX9 treated HT1080 cells.

FIG. 8 shows growth inhibition of normal mammary epithelial cells and breast cancer cell lines by three anticancer drugs and SNX9.

FIG. 9 shows growth inhibition of normal fibroblasts and different tumor cell lines by three anticancer drugs, SNX9 and SNX9-1.

FIG. 10 shows the effects of SNX9-1 and SNX14 on the expression of p21-responsive genes in HT-1080 cells, with or without p21 induction.

FIG. 11 shows a proposed mechanism of action of SNX compounds on the CDK Inhibitor (CDKI) pathway.

FIG. 12 shows the effects of a number of SNX compounds on kinase activity of Cyclin/CDK complexes.

FIG. 13 shows growth inhibition of transformed and untransformed human fibroblasts by different CDK inhibitors.

FIG. 14 shows inhibition of CDK3 mRNA expression in HT1080 cells transduced with a mixture of three CDK3-targeting shRNA lentiviruses.

FIG. 15 shows effects of CDK3-targeting shRNA lentiviruses on cell growth.

FIG. 16 shows SAGE anatomical view of CDK3 expression in normal and tumor cells and tissues.

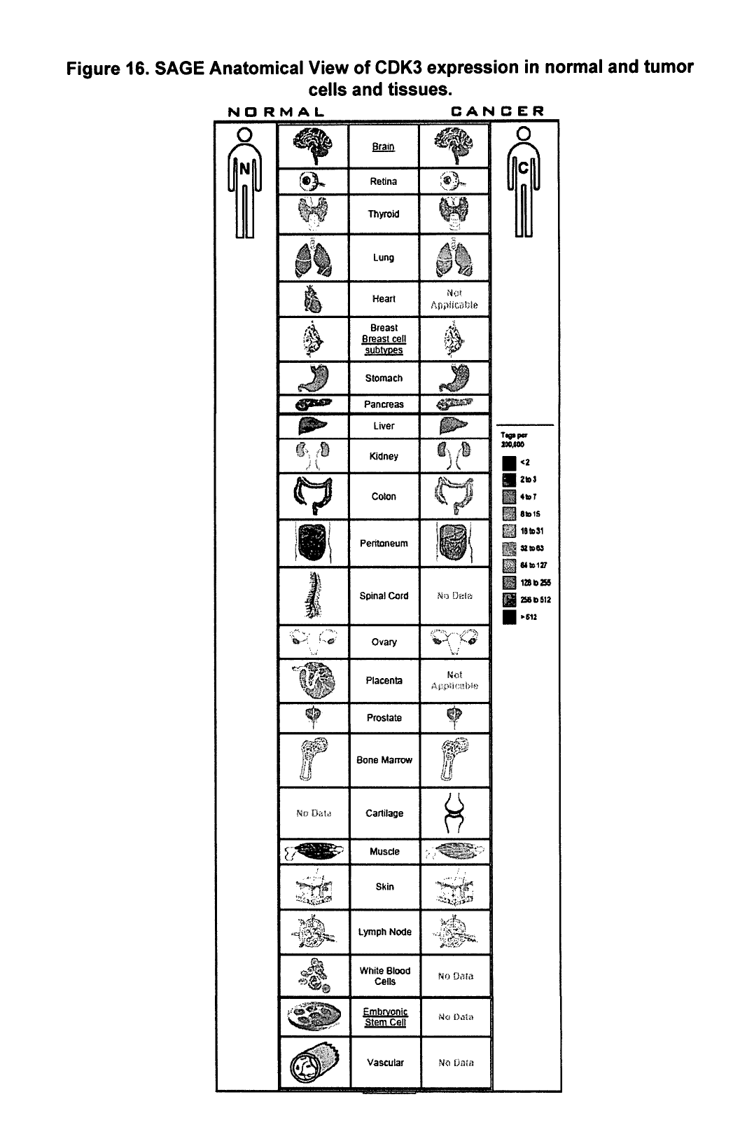

FIG. 17 shows HCT116 xenograft tumor growth in nude mice treated with vehicle, SNX9 or SNX9-1.

### DETAILED DESCRIPTION OF THE PREFERRED EMBODIMENTS

The present inventors have identified several compounds with the following CDK-related activity. These compounds inhibit the CDKI pathway, defined as the induction of transcription of multiple genes in response to the expression of a CDK inhibitor protein, such as p21^{Waf1}. The CDKI pathway inhibitors, designated SNX9 class compounds, have a desirable ability to inhibit the growth of different types of tumor cells preferentially to normal cells.

The invention provides new methods for specifically inhibiting tumor cell growth. The invention further provides new and specific inhibitors of tumor cell growth, as well as means for discovery of additional such inhibitors. The present inventors have surprisingly discovered that Cyclin-Dependent Kinase 3 (CDK3) is specifically required for tumor cell growth, in contrast to other members of the CDK family.

The references cited herein reflect the level of knowledge in the field and are hereby incorporated by reference in their entirety. Any conflicts between the teachings of the cited references and this specification shall be resolved in favor of the latter.

In a first aspect, the invention provides a method for selectively inhibiting tumor cell growth comprising selectively inhibiting in a tumor cell cyclin-dependent kinase 3 (CDK3). For purposes of the invention, “selectively inhibiting tumor cell growth” means inhibiting the growth of fully transformed or partially transformed cells, relative to untransformed cells. “Selectively inhibiting CDK3” means inhibiting CDK3 to a greater extent than inhibiting other cyclin dependent kinases (CDKs), including CDK1, CDK2, and CDK4/CDK6. “Inhibiting a cyclin dependent kinase (CDK)” means reducing the activity and/or expression of a CDK. Preferred methods of inhibiting CDK3 include, without limitation, contacting CDK3 (preferably in a tumor cell) with a small molecule inhibitor of CDK3 activity, or a dominant negative mutant of CDK3, such as a CDK3 protein with some but not all of its protein- or substrate-interactive domains inactivated or a genetic suppressor element (GSE) that encodes a fragment of the CDK3 protein, which interferes with the CDK3 activity. A preferred small molecule specific inhibitor of CDK3 is an SNX9 class compound. Contacting CDK3 with its dominant negative mutant includes expressing the dominant negative mutant via transfection with a virus or a vector expressing the dominant negative mutant, or contacting CDK3-expressing cells with a peptide encoded by the GSE. Additional preferred methods include contacting a cell with an inhibitor of CDK3 gene expression, including without limitation, a short hairpin RNA (shRNA), a small inhibitory RNA (siRNA), an antisense nucleic acid (AS) and a ribozyme. “Contacting a cell with an inhibitor of CDK3 gene expression” includes exogenously providing to a cell an inhibitor of CDK3 gene expression, as well as expressing an inhibitor of CDK3 gene expression in a cell. Expressing an inhibitor of gene expression in a cell is conveniently provided by transfection with a virus or a vector expressing such an inhibitor.

In a second aspect, the invention provides a method for identifying a specific inhibitor of tumor cell growth, the method comprising contacting an in vitro complex of a purified cyclin and CDK3 under conditions in which the complex of purified cyclin and CDK3 is capable of exhibiting kinase activity with a candidate inhibitor of such activity, and measuring the kinase activity of such complex in the presence or absence of such candidate compound. According to this aspect of the invention, a complex of a CDK3-interacting cyclin or cyclin-related molecule, such as cyclin E, cyclin C, cyclin A or CABLES1 and CDK3 is used. For comparison (control) experiments, a complex of CDK1, CDK2, CDK4 or CDK6 with a cyclin interacting with the corresponding CDK is used. A candidate compound is regarded as a specific inhibitor of tumor cell growth if (1) the activity of a cyclin/CDK3 complex in the presence of the candidate inhibitor is lower than the kinase activity of the cyclin/CDK3 complex in the absence of the candidate inhibitor, and preferably if (2) the candidate inhibitor inhibits the activity of a cyclin/CDK3 complex to a greater extent than the kinase activity of a complex of CDK1, CDK2, CDK4, or CDK6 with a cyclin interacting with the corresponding CDK. “The absence of the candidate inhibitor” means that either no small molecule CDK inhibitor is present, or a CDK inhibitor which is known to not be an inhibitor of a CDK is present. As a positive control, the inhibitory activity of the candidate inhibitor may be compared with the inhibitory activity of a known specific inhibitor of CDK3, such as an SNX9 class compound.

In a third aspect, the method provides specific inhibitor compounds of CDK3, including such compounds identified by the method according to the second aspect of the invention.

Some preferred compounds according to the invention have the structure I:

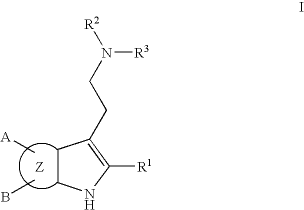

including free acids, salts, or prodrugs thereof;
wherein R¹, R² and R³ are each independently selected from hydrogen, C1-C6 alkyl, cycloalkyl, alkenyl, O-alkyl, dialkylaminoalkyl, aryl and heteroaryl, or R² and R³ may together form a ring of 3-6 atoms, which may include one or more heteroatom and/or 1 or more double bond; A and B are each independently selected from hydrogen, O-alkyl, N-alkyl, and an electron-withdrawing group, provided, however, that at least one of A or B is an electron withdrawing group; and Z is a 4-10 atom ring structure, which may contain 0-3 heteroatoms, and may contain 1 or more double bonds.

Some preferred compounds according to the invention have the structure II:

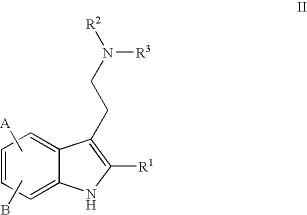

including free acids, salts, or prodrugs thereof;
wherein R¹, R² and R³ are each independently selected from hydrogen, C1-C6 alkyl, cycloalkyl, alkenyl, O-alkyl, dialkylaminoalkyl, aryl and heteroaryl, or R² and R³ may together form a ring of 3-6 atoms, which may include one or more heteroatom and/or 1 or more double bond; and A and B are each independently selected from hydrogen, O-alkyl, N-alkyl and an electron-withdrawing group, provided, however, that at least one of A or B is an electron withdrawing group.

In some preferred embodiments of structures I and II, R¹ is hydrogen or methyl, and R² and R³ are independently selected from hydrogen, methyl and C2-C6 alkyl or alkenyl.

In some preferred embodiments, one or more electron-withdrawing group may be selected from halogen, a nitrogen-containing group, or an oxygen-containing group. In some preferred embodiments, one or more halogen is not fluorine, and may be preferably selected from chlorine and bromine.

In some preferred embodiments, the compound used in the methods according to the invention is selected from:

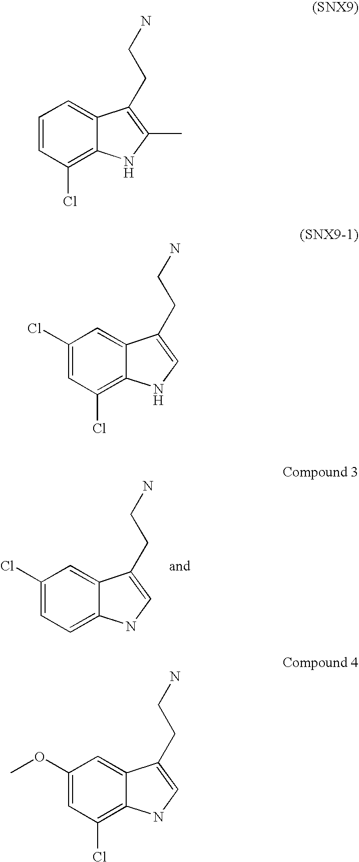

including free acids, salts or prodrugs thereof.

In a fourth aspect, the invention provides a method for treating a patient having a tumor with compounds that reduce or prevent the induction of transcription by cyclin-dependent kinase inhibitors. The method according to this aspect of the invention comprises administering to a patient having a tumor a compound according to the invention. Cyclin-dependent kinase inhibitors (CDKI) induce transcription of genes through the formation of a complex between a cyclin, a cyclin-dependent kinase (CDK) and the cyclin-dependent kinase inhibitor (e.g., p21, p16, p27). Compounds that reduce or prevent CDKI induced transcription act by interfering with such complex formation, destabilizing the complex, or otherwise rendering the complex inoperative. Some compounds that selectively interfere with tumor cell growth act directly on CDK3.

In a fifth aspect, the invention provides a method for inhibiting the Cyclin-Dependent Kinase Inhibitor (CDKI) pathway downstream of the CDKI proteins and upstream of genes that are transcriptionally activated by the CDKI pathway. This method may have a variety of clinical applications in chemoprevention and therapy of different age-related diseases. In preferred embodiments, the method according to the invention comprises contacting a cell with a small molecule inhibitor having the structure (I) or (II).

In the methods for treatment according to the invention, the compounds and other inhibitors described above may be incorporated into a pharmaceutical formulation. Such formulations comprise the compound, which may be in the form of a free acid, salt or prodrug, in a pharmaceutically acceptable diluent, carrier, or excipient. Such formulations are well known in the art and are described, e.g., in Remington's Pharmaceutical Sciences, 18th Edition, ed. A. Gennaro, Mack Publishing Co., Easton, Pa., 1990.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art.

As used herein, the term “pharmaceutically acceptable salts” refers to salts that retain the desired biological activity of the above-identified compounds and exhibit minimal or no undesired toxicological effects. Examples of such salts include, but are not limited to, salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, palmoic acid, alginic acid, polyglutamic acid, naphthalenesulfonic acid, naphthalenedisulfonic acid, methanesulfonic acid, p-toluenesulfonic acid, polygalacturonic acid, and the like. The compounds can also be administered as pharmaceutically acceptable quaternary salts known by those skilled in the art, which specifically include the quaternary ammonium salt of the formula —NR+Z-, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

Administration of the pharmaceutical formulations in the methods according to the invention may be by any medically accepted route, including, without limitation, parenteral, oral, sublingual, transdermal, topical, intranasal, intratracheal, or intrarectal. In certain preferred embodiments, compositions of the invention are administered parenterally, e.g., intravenously in a hospital setting. In certain other preferred embodiments, administration may preferably be by the oral route.

### EXAMPLES

The following examples are intended to further illustrate certain particularly preferred embodiments of the invention and are not intended to limit the scope of the invention.

### Example 1

### Identification of CDKI Pathway Inhibitors

As described in a co-pending patent application PCT/US06/0 1046, we have developed a high-throughput screening (HTS) procedure for compounds inhibiting the CDKI pathway. This procedure utilizes a highly sensitive reporter cell line that was generated by infecting HT1080 p21-9 cells, a derivative of HT1080 fibrosarcoma cells that express p21 from a promoter induced by a physiologically neutral β-galactoside IPTG (isopropyl-β-thio-galactoside) with a lentiviral vector that expresses Green Fluorescent Protein (GFP) from the CDKI-inducible cytomegalovirus (CMV) promoter, followed by subcloning of GFP positive cells and monitoring the induction of GFP expression by IPTG. A cell line showing approximately 10-fold increase in GFP upon the addition of IPTG was used for HTS in a 96-well format.

This reporter line was used to screen two diversified small-molecule libraries developed by ChemBridge Corp., Microformat 04 and DiverSet, each comprising 50,000 compounds. These diversified libraries were rationally chosen by ChemBridge by quantifying pharmacophores in a collection of >500,000 drug-like molecules, using a version of Chem-X software to maximize the pharmacophore diversity. The Microformat 04 collection was designed to complement the chemical space covered by the older DiverSet library. ChemBridge libraries have been successfully used by numerous industrial and academic researchers, in a variety of cell-based and cell-free assays. The ChemBridge libraries were screened at 20 μM concentration, a conventional concentration for cell-based screening of these libraries. 62 of 100,000 ChemBridge compounds were identified by HTS and verified as inhibiting the induction of CMV-GFP expression in response to p21. This low hit rate (0.06%) indicates a high selectivity of our assay. SNX9, SNX9-1 and compounds 3-4, specifically shown above, showed anti-CDKI pathway activity in the reporter assay.

### Example 2

### Effects of Compounds on CDKI-Induced Transcription

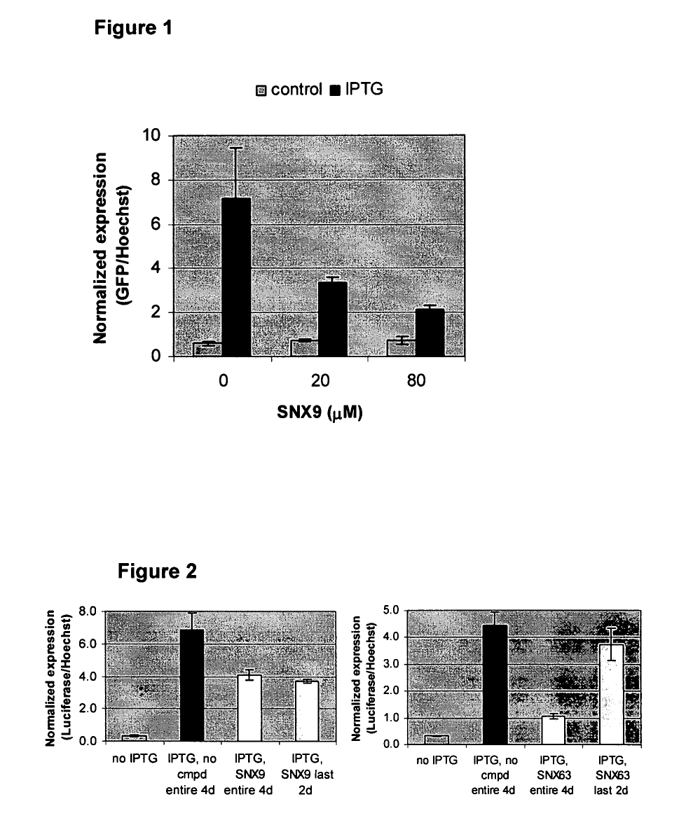

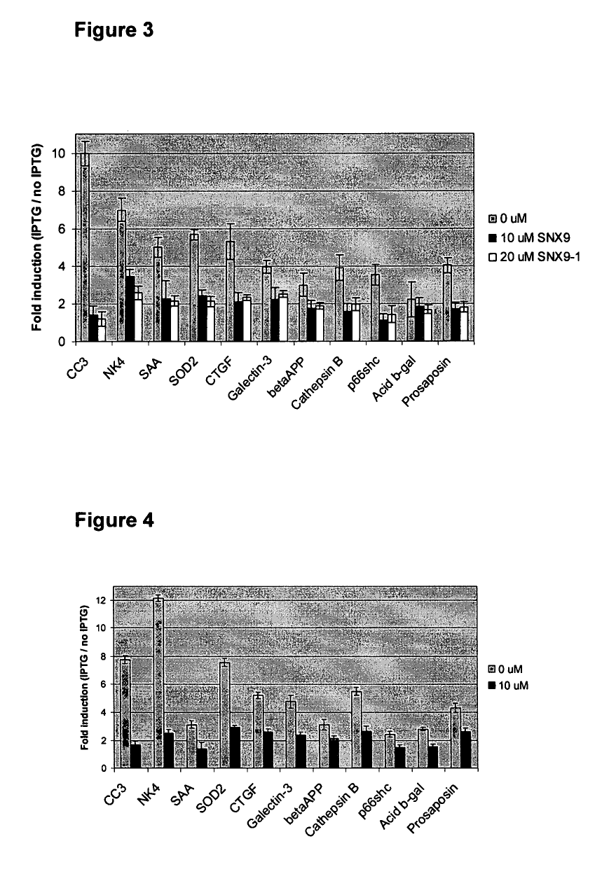

FIG. 1 shows the effect of SNX-9 on normalized GFP expression in the reporter cell line, in the presence or in the absence of IPTG (the p21 inducer). The compound shows pronounced inhibition of transcription by p21, but it does not inhibit promoter function when p21 is not induced, indicating that its transcriptional effect is specific for CDKI-induced transcription. Similar results were obtained with SNX9-1, and Compound 3 and 4. The experiment in FIG. 2 shows that SNX-9 can also reverse p21-induced transcription. In this experiment, HT1080 p21-9 cells that express firefly luciferase from a CDKI-responsive promoter of cellular NK4 gene were cultured with IPTG for two days, which is sufficient for near-maximal induction of NK4 (Poole et al., supra). The addition of 20 μM SNX-9 decreased the induction of NK4-luciferase by p21 not only when the compound was added simultaneously with IPTG but also when added after two days of IPTG treatment, indicating that the compound not only prevents but also reverses CDKI-induced transcription (as a negative control, FIG. 2 shows that an unrelated compound SNX63 inhibited transcription only when added simultaneously with IPTG but not two days later). The ability to reverse CDKI-induced transcription suggests that drugs derived from SNX9 may be useful not only for chemoprevention but also for therapeutic applications for diseases involving the CDKI pathway.

We determined whether this class of compounds inhibits the effect of CDKI not only on artificial promoter-reporter constructs but also on CDKI-responsive endogenous genes. For this purpose, we developed real-time RT-PCR (Q-PCR) assays for measuring RNA levels of eleven CDKI-responsive genes. This assay uses a 96-well TurboCapture RNA extraction kit (Qiagen), in which oligo(dT) is covalently bound to the surface of the wells to allow mRNA isolation from cell lysate and cDNA synthesis in the same wells. 5 units/μl of SuperScript III reverse transcriptase (Invitrogen) was added to the wells for 1 hr for cDNA synthesis at 50° C., and 2 μl of the resulting cDNA was then used for Q-PCR analysis using SYBR Green PCR Master Mix (ABI) with ABI 7900HT Q-PCR machine. Primers used to amplify specific gene products for the corresponding genes and for β-actin (control) are listed in Table 1.

TABLE 1
Sequence of primers used in Q-PCR

| | | | |
|---|---|---|---|
| | | | Product |
| | | | size |
| Gene | Sense (5′-3′) | Antisense (5′-3′) | (bp) |

| | | | |
|---|---|---|---|
| Acid β- | CGATCGAGCATATGTTGCTG | AGTTCACACGTCCCATGT | 134 |
| galactosidase | | | |
| Complement C3 | ATCCGAGCCGTTCTCTACAA | CTGGTGACGCCTCTTGGT | 111 |
| Connective | GGAGTGGGTGTGTGACGAG | CCAGGCAGTTGGCTCTAATC | 116 |
| Tissue | | | |
| Growth | | | |
| Factor | | | |
| Galectin-3/ | GGAGCCTACCCTGCCACT | CCGTGCCCAGAATTGTTATC | 118 |
| Mac-2 | | | |
| NK4 | CACAGCACCAGGCCATAGA | TCTGCCAGGCTCGACATC | 85 |
| p66shc | TTCGAGTTGCGCTTCAAAC | TCAGGTGGCTCTTCCTCCT | 116 |
| SAA | GTTCCTTGGCGAGGCTTT | CCCCGAGCATGGAAGTATT | 105 |
| Prosaposin | GCTTCCTGCCAGACCCTTAC | CCAATTTTCAAGCACACGAA | 118 |
| SOD2 | CCTAACGGTGGTGGAGAACC | CAGCCGTCAGCTTCTCCTTA | 94 |
| βAPP | GGACCAAAACCTGCATTGAT | CTGGATGGTCACTGGTTGG | 113 |
| β-Actin | CTTCCTGGGCATGGAGTC | TGTTGGCGTACAGGTCTTTG | 95 |

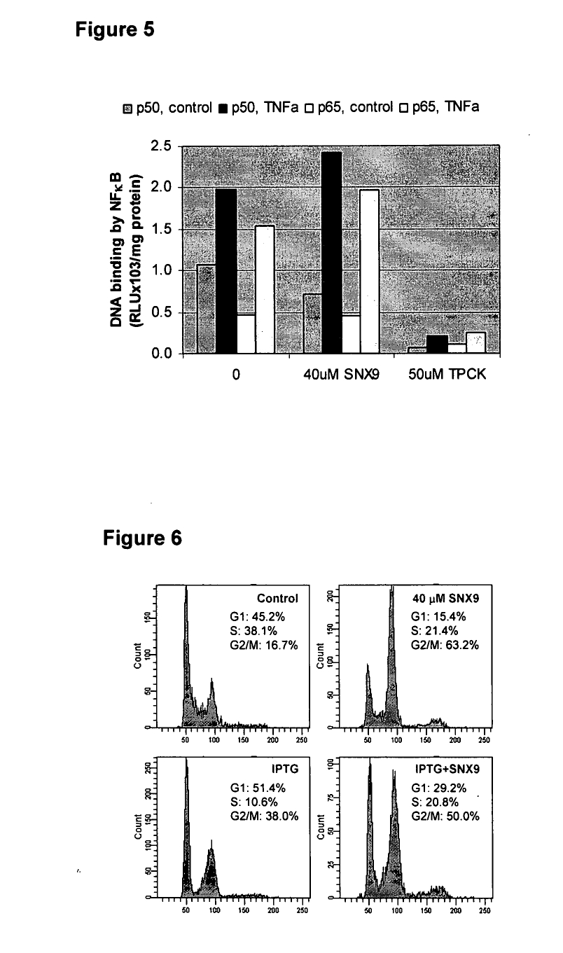

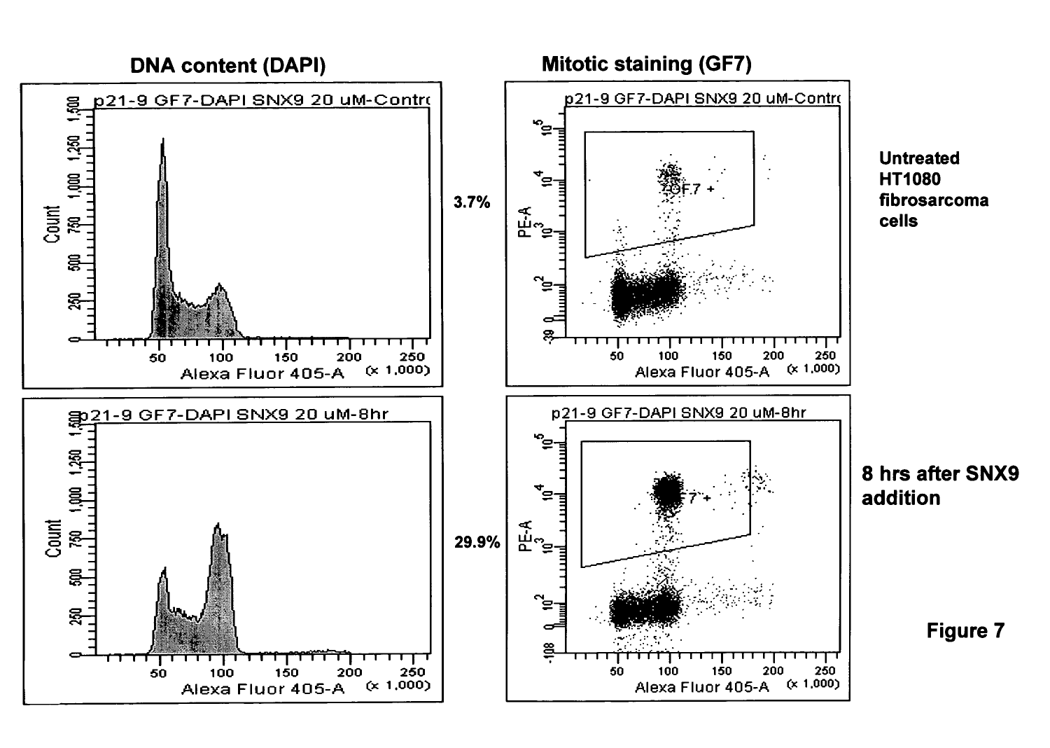

FIG. 3 shows the effects of SNX9 and SNX9-1 on the induction of these genes in HT1080 cells with IPTG-inducible expression of p21, with the results expressed as the ratio of RNA levels for each gene in the presence and in the absence of IPTG (β-actin, expression of which is not affected by CDKI, was used as a normalization standard). FIG. 4 shows the same analysis for the effects of SNX9 in HT1080 cells with IPTG-inducible expression of p16. These compounds partially inhibit the induction of all the tested genes in either p21- or p16-arrested cells. This effect argues that the molecular target of these compounds is not a specific CDKI but rather a common downstream mediator of the transcription-inducing effects of different CDKI.

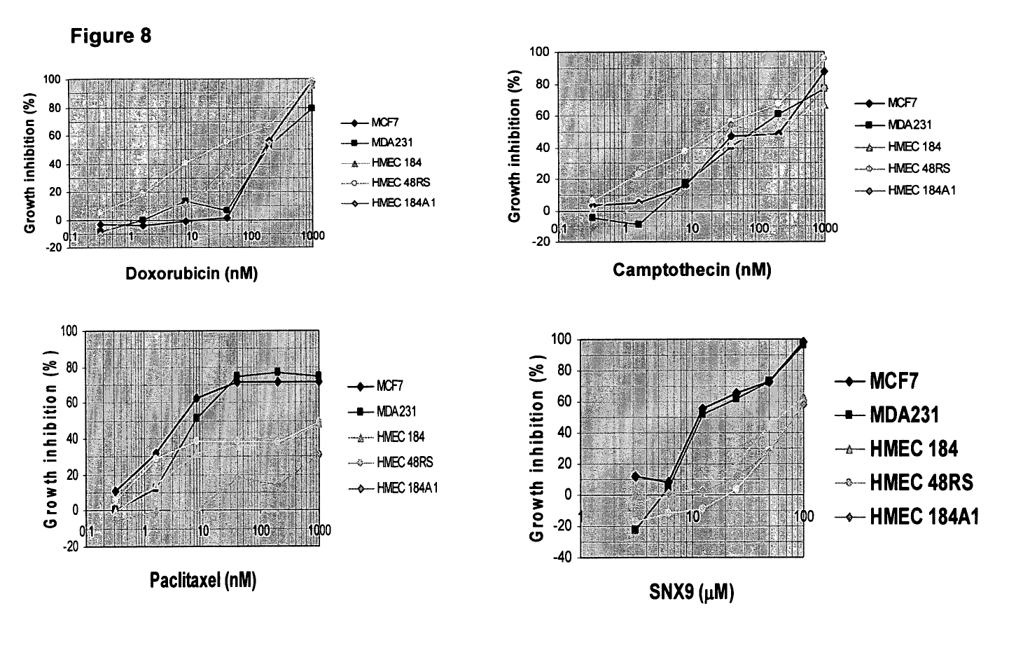

We also tested if these compounds could act as the inhibitors of NFκB, by measuring cellular levels of p50 or p65 subunits binding oligonucleotides containing NFκB consensus binding site, using ACTIVE MOTIF TransAM™ NFκB p65 Chemi and NFκB p50 Chemi Transcription Factor Assay Kits. As shown in FIG. 5, SNX9 has no significant effect on either TNFα-induced or basal NFκB activity, in contrast to NFκB inhibitor TPCK (positive control), which completely blocks NFκB activity in these assays.

### Example 3

### Tumor-Specific Growth Inhibition by SNX9-Class Compounds

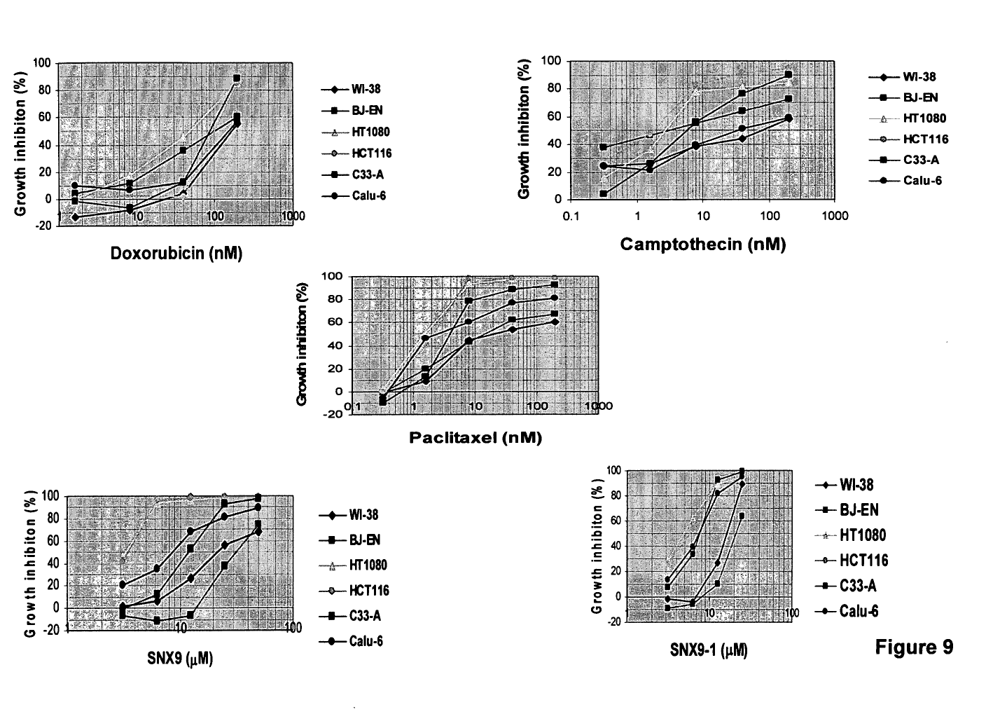

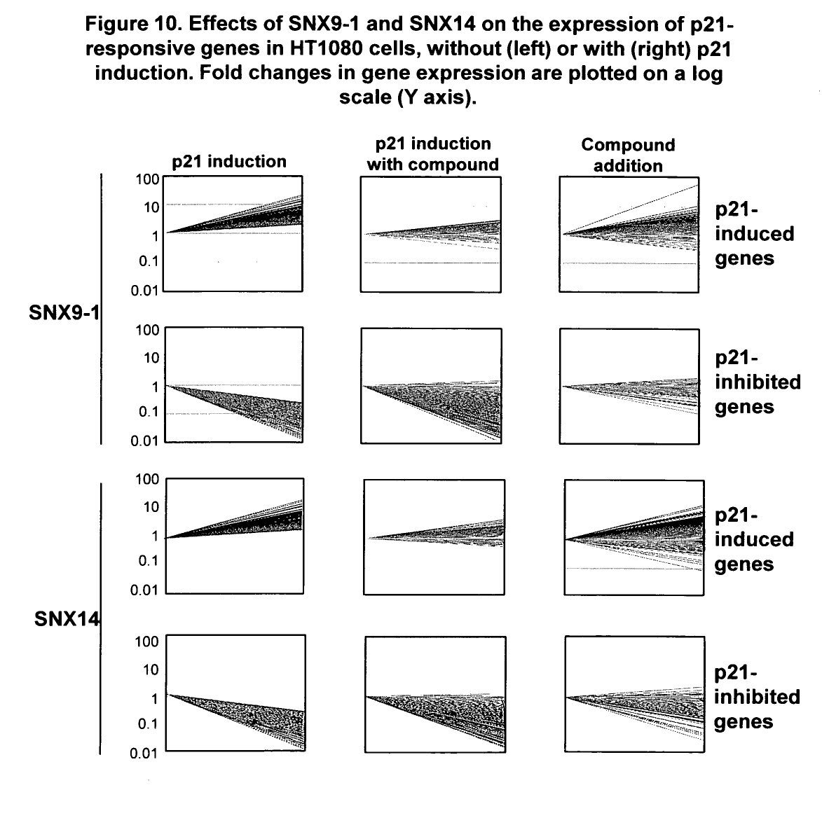

Surprisingly, the majority of 62 compounds identified in the screen for CDKI pathway inhibitors showed pronounced growth inhibition of the HT1080-derived reporter cell line. In the case of SNX9, SNX9-1 and Compounds 3 and 4, cell growth inhibition was associated with the induction of both cell death (as detected microscopically by cell detachment) and cell cycle arrest. The latter is illustrated in FIG. 6, which shows FACS analysis of DNA content of HT1080 p21-9 cells that were either untreated, or treated with SNX9 alone, with IPTG (that induces p21) alone, or with a combination of SNX9 and IPTG. SNX9 treatment induced a pronounced increase in the G2/M fraction. Staining with an antibody GF7 specific for mitotic cells (Rundle et al., J. Biol. Chem. 276: 48231-48236, 2001) shows an increase in the mitotic fraction of SNX9-treated cells, indicating that the G2/M arrest by SNX9 occurs largely or exclusively in mitosis (FIG. 7). p21 induction by IPTG arrested cells both in G1 and G2. The combination of p21 and SNX9-class compounds leads to both G1 and G2/M arrest, at the levels expected for the combined effects of p21 and SNX9 (FIG. 6). This analysis indicates that SNX9-class compounds inhibit the cell cycle in G2/M and do not interfere with p21-induced G1 arrest. Hence, SNX9-class compounds do not block the essential function of CDKI as cell cycle inhibitors.

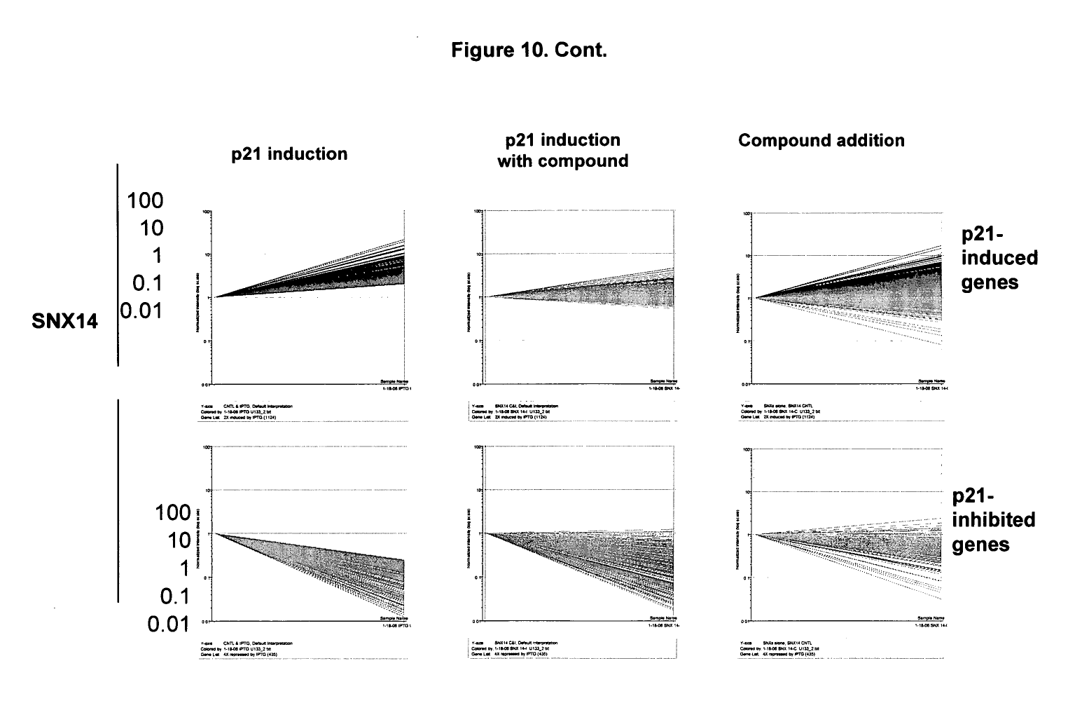

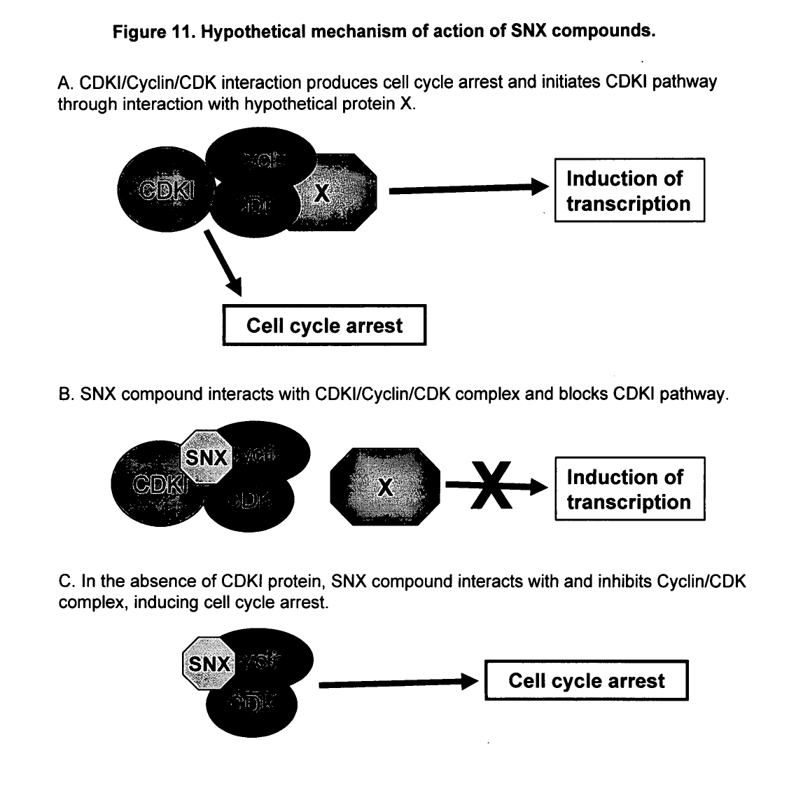

We have compared the growth-inhibitory effects of SNX9-class compounds on different tumor and normal cells. FIG. 8 and FIG. 9 show the results of growth inhibition assays carried out with various cell lines, using different doses of SNX9, SNX9-1 and three well-known anticancer drugs, doxorubicin (Adriamycin), camptothecin and paclitaxel (Taxol). The assays were carried out in 96-well plates, in triplicates; the plated cell numbers for each cell line were determined in preliminary experiments to assure exponential growth over the 3-day period of the assay. The cell numbers were measured by staining DNA of attached cells with Hoechst 33342, and the results for each dose were expressed as the decrease in cell number relative to untreated cells. For some of the tumor/normal cell line combinations, the assays were repeated using CellTiter-Glo viability assay (Promega), with the same results. FIG. 8 compares the effects of the compounds on three primary cultures of human mammary epithelial cells (HMEC) and two breast carcinoma cell lines (MCF-7 and MDA231). Doxorubicin and camptothecin provide no discrimination between normal and transformed mammary cells, but clear tumor selectivity is apparent with taxol and SNX9. FIG. 9 compares the effects of compounds on primary (WI-38) and hTERT-immortalized normal BJ fibroblasts (BJ-EN) with their effects on HT1080 fibrosarcoma and three carcinoma cell lines, HCT116 colon carcinoma, C33-A cervical carcinoma, and Calu-6 lung carcinoma. In this set, SNX9, SNX9-1 and taxol inhibited all the tumor cells to a greater extent than normal fibroblasts, but doxorubicin and camptothecin showed no selectivity. SNX9 was especially potent against HCT116 and HT1080 cells, where it produced close to 100% inhibition at a 5 μM concentration that had no effect on normal cells, a selectivity unmatched with any anticancer drugs.

These results demonstrate that SNX9-class compounds exhibit the essential effect expected for CDKI pathway inhibitors, blocking the induction and reversing CDKI-induced transcription, and also show pronounced tumor-specific growth-inhibitory activity. SNX9-class compounds therefore constitute prototypes of drugs that are likely to be useful for chemoprevention and therapy of cancer.

### Example 4

### Microarray Analysis Suggests CDKI-Like Activity of CDKI Pathway Inhibitors

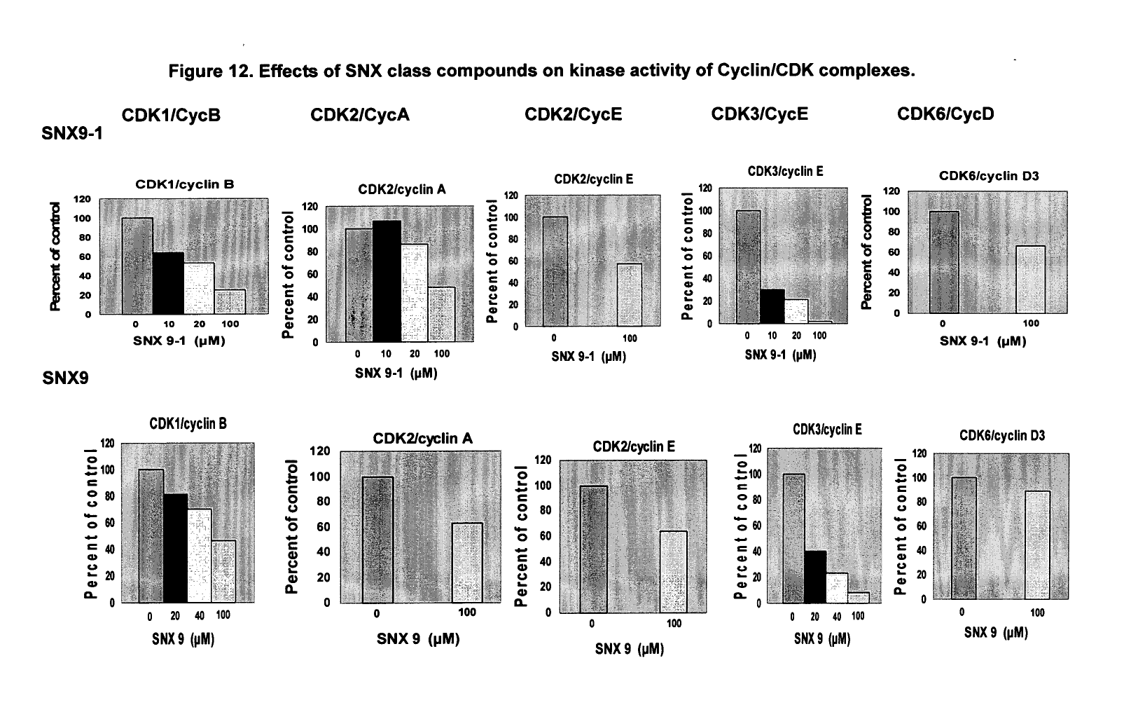

HT1080 p21-9 cell line, which carries IPTG-inducible CDKI p21, was either untreated, or treated for 72 hrs with 100 μM IPTG (which induces p21), or with CDKI pathway inhibitors SNX9-1 (20 μM, SNX9 family) or SNX14 (80 μM, unrelated to SNX9) alone or in combination with IPTG. RNA was extracted after each treatment and used for hybridization with Affymetrix U133 2.0 Plus microarrays, containing 56,000 probe sets corresponding to essentially all the human genes. The microarray data were analyzed using Gene Spring software (Agilent). FIG. 10 displays changes in the expression of two groups of genes. The first group (p21-induced genes, top panels) represents 1124 probe sets corresponding to genes that were induced at least 2-fold upon treatment with p21-inducing IPTG. The second group (p21-inhibited genes, bottom panels) represents 435 probe sets corresponding to genes that were inhibited at least 4-fold upon IPTG treatment. Each panel shows fold changes in gene expression (log scale), from cells that either were not treated (left) or were treated (right) with the indicated compound. The left panels show the response of the two groups of genes to IPTG (p21 induction) in the absence of SNX9-class compounds, with the first group induced and the second group inhibited by IPTG. The middle panels show the response of the same genes to IPTG in the presence of SNX9-1 or SNX14 (added with or without IPTG). Both the induction and the inhibition of gene expression by IPTG appear much reduced in the presence of SNX9-1 or SNX14, as expected from their activity as CDKI pathway inhibitors.

In particular, we found that 9 of 14 genes, identified by Stein et al., supra, as markers of cancer intractability were induced by p21 in this system, but SNX9-1 and SNX14 inhibited their induction (Table 2). This result indicates potential utility of CDKI pathway inhibitors for diminishing cancer intractability.

TABLE 2
Effects of SNX14 and SNX9-1 on the induction of genes
associated with cancer intractability by IPTG-induced p21

| | | Fold induction by IPTG | | |
|---|---|---|---|---|
| Affymetrix ID | Gene Name | Control | SNX14 | SNX9-1 |
| 203828_s_at | NK4 | 7.22 | 1.68 | 1.60 |
| 208949_s_at | LGALS3 | 3.74 | 1.40 | 1.33 |
| 204981_at | SLC22A18 | 2.46 | 1.56 | 1.34 |
| 209008_x_at | KRT8 | 1.97 | 0.86 | 0.81 |
| 211043_s_at | CLTB | 1.32 | 1.00 | 0.95 |
| 218148_at | FLJ13111 | 1.27 | 1.13 | 0.98 |
| 212071_s_at | SPTBN1 | 1.24 | 1.09 | 1.24 |
| 212063_at | CD44 | 1.21 | 0.89 | 1.03 |
| 226765_at | SPTBN1 | 1.21 | 1.06 | 1.09 |

The right panels of FIG. 10 show the response of the same two groups of genes to SNX9-1 or SNX14, in the absence of IPTG. Remarkably, most of p21-induced genes are induced by SNX9-1 and SNX14 and most of p21-inhibited genes are inhibited by SNX9-1 and SNX14. Although the effects of these compounds are weaker than the effects of p21-inducing IPTG, they indicate that CDKI pathway inhibitors can partially mimic the effect of CDKI p21 on gene expression.

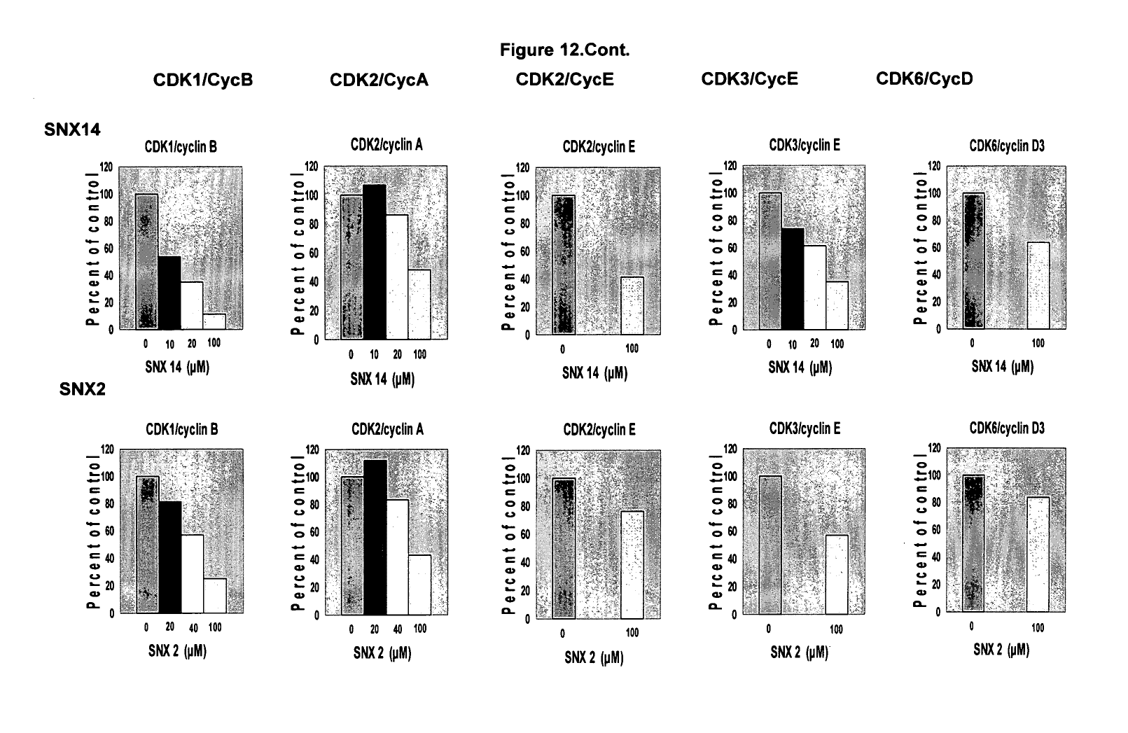

This finding suggested the following hypothesis (FIG. 11). According to this hypothesis, formation of a complex between a CDKI protein (e.g. p21), a cyclin and a CDK leads both to cell cycle arrest (due to CDK inhibition) and to the activation of the CDKI transcriptional pathway due to the interaction of the CDKI/Cyclin/CDK complex with an as yet undefined regulatory protein X (FIG. 11A). SNX compounds physically interact with the CDKI/Cyclin/CDK complex and prevent its interaction with protein X, thereby blocking the CDKI pathway (FIG. 11B). In the absence of the CDKI protein, SNX compounds still bind to Cyclin/CDK complexes. This binding partially mimics the transcriptional effects of the CDKI protein (as detected by microarray analysis), and also inhibits CDK activity, which would explain cell cycle arrest produced by CDK inhibitors (FIG. 11C).

### Example 5

### CDKI Pathway Inhibitors Have Direct CDK Inhibitor Activity

To test the above hypothesis, we determined whether several CDKI pathway inhibitors, including two SNX9 class compounds (SNX9 and SNX9-1), two compounds of a different structural class (SNX14 and SNX2) and another unrelated inhibitor (SNX35), inhibit the kinase activity of different complexes formed in vitro by purified cyclin/CDK complexes. This analysis was carried out as a service by Upstate Biotechnology, Inc.

CDK1/cyclinB (h)

In a final reaction volume of 25 μl, CDK1/cyclinB (h) (5-10 μU) is incubated with 8 mM MOPS pH 7.0, 0.2 mM EDTA, 0.1 mg/ml histone H1, 10 mM MgAcetate and [γ-33P-ATP] (specific activity approx. 500 cpm/pmol, 45 μM concentration). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK2/cyclinA (h)

In a final reaction volume of 25 μl, CDK2/cyclinA (h) (5-10 mU) is incubated with 8 mM MOPS pH 7.0, 0.2 mM EDTA, 0.1 mg/ml histone H1, 10 mM MgAcetate and [γ-33P-ATP] (specific activity approx. 500 cpm/pmol, 45 μM concentration). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK2/cyclinE (h)

In a final reaction volume of 25 μl, CDK2/cyclinE (h) (5-10 mU) is incubated with 8 mM MOPS pH 7.0, 0.2 mM EDTA, 0.1 mg/ml histone H1, 10 mM MgAcetate and [γ-33P-ATP] (specific activity approx. 500 cpm/pmol, 120 μM concentration). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK3/cyclinE (h)

In a final reaction volume of 25 μl, CDK3/cyclinE (h) (5-10 mU) is incubated with 8 mM MOPS pH 7.0, 0.2 mM EDTA, 0.1 mg/ml H1, 10 mM MgAcetate and [γ-33P-ATP] (specific activity approx. 500 cpm/pmol, 200 μM concentration). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

CDK6/cyclinD3 (h)

In a final reaction volume of 25 μl, CDK6/cyclinD3 (h) (5-10 mU) is incubated with 8 mM MOPS pH 7.0, 0.2 mm EDTA, 0.1 mg/ml H1, 10 mM MgAcetate and [γ-33P-ATP] (specific activity approx. 500 cpm/pmol, 200 μM concentration). The reaction is initiated by the addition of the MgATP mix. After incubation for 40 minutes at room temperature, the reaction is stopped by the addition of 5 μl of a 3% phosphoric acid solution. 10 μl of the reaction is then spotted onto a P30 filtermat and washed three times for 5 minutes in 75 mM phosphoric acid and once in methanol prior to drying and scintillation counting.

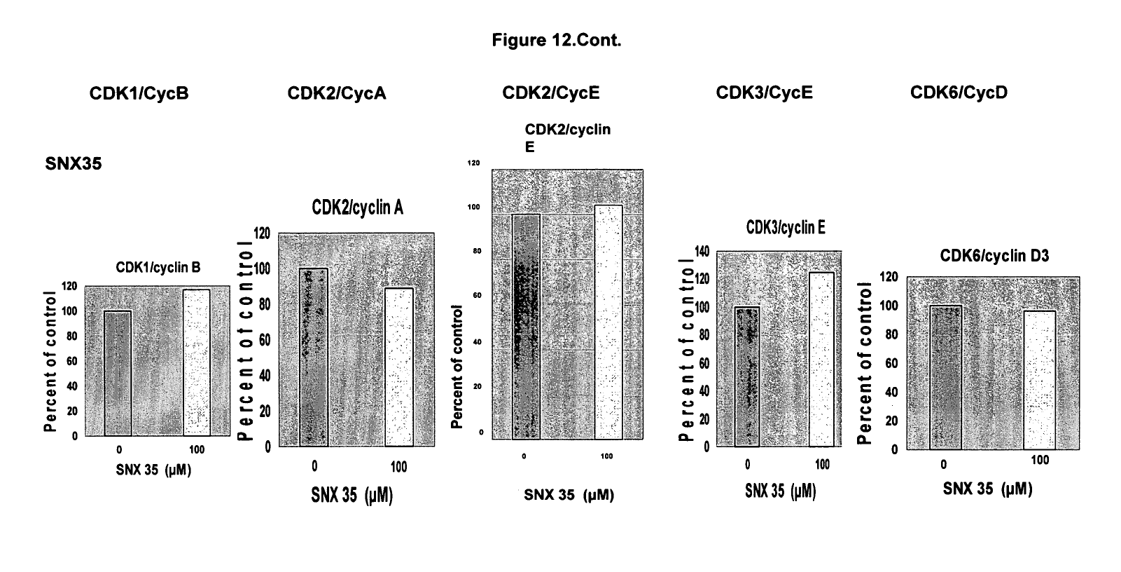

At the first step, all the compounds were tested at 100 μM concentration. At the second step, Cyclin/CDK complexes that showed significant sensitivity to the compounds were re-tested with two lower compound concentrations (20 and 40 μM for SNX9 and SNX2, 10 and 20 μM for more potent compounds SNX9-1 and SNX14). FIG. 12 shows the results of these assays, displayed as relative kinase activity of different cyclin/CDK complexes in the presence of SNX compounds (the kinase activity in the absence of the compounds is taken as 100%). Only SNX35 had no effect on cyclin/CDK activities. SNX9-1, SNX9, SNX2 and SNX14 all inhibited the kinase activities of CDK1 (CDC2), CDK2, CDK3 and CDK6. For SNX14 and SNX2, the relative efficacy of inhibition was CDK1>CDK2˜CDK3>CDK6. For SNX9 and SNX9-1, the order was CDK3>CDK1>CDK2>CDK6 (FIG. 12). Hence, some CDKI pathway inhibitors have CDK inhibitor activity, and SNX9 class compounds (which show the highest tumor selectivity in their growth inhibitory effect) preferentially inhibit CDK3.

### Example 6

### Growth-Inhibitory Effect of Selective Inhibitors of CDK1 CDK2 or CDK4 Does Not Show the Tumor Selectivity of SNX9 Class Compounds

We have previously demonstrated that SNX9 class compounds preferentially inhibit the growth of many different tumor cell lines relative to normal cells (human fibroblasts and normal mammary epithelial cells). To determine if tumor-specific growth inhibitory activity of SNX9 class compounds could be due to the inhibition of a specific CDK, we took advantage of the availability of selective inhibitors for some of the CDK proteins. Among these enzymes, CDK1, CDK2 and CDK4/CDK6 (the latter two CDKs are closely related to each other and interact with the same class of cyclins), have been used as targets for developing specific inhibitors, with potential anticancer activity. In contrast, CDK3 has not been used as a target for developing selective inhibitors. We have obtained commercially available selective inhibitors of CDK1 (CGP74514A) (Calbiochem Cat. No. 217696), CDK2 (CVT-313) (Calbiochem Cat. No. 238803), and CDK4 (NSC 625987) (Calbiochem Cat. No. 219477), and tested them, along with a broad-specificity CDK inhibitor flavopiridol, and with SNX14, SNX9 and SNX9-1, for growth inhibition of normal and transformed fibroblasts.

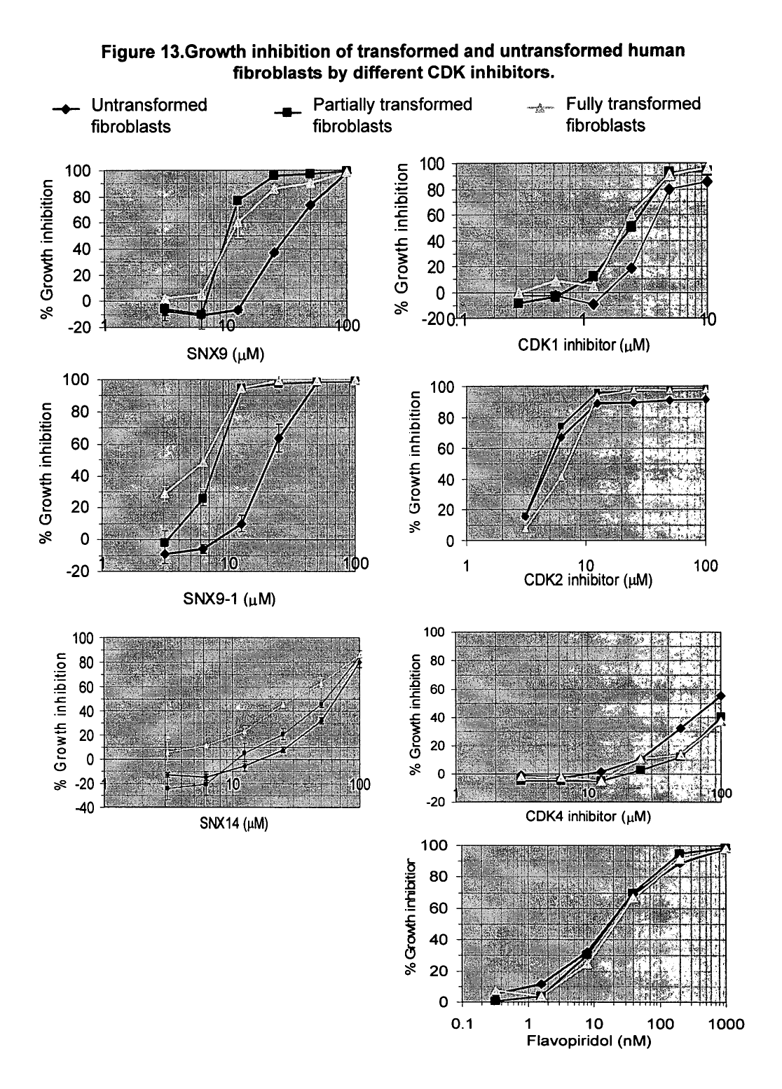

This analysis was done using a set of three isogenic human fibroblast cell lines with increasing degrees of neoplastic transformation (immortal but untransformed, partially transformed, and fully transformed). The dose-dependent growth inhibition assays were carried out in 96-well plates, in triplicate; the plated cell numbers for each cell line were determined in preliminary experiments to assure exponential growth over the 3-day period of the assay. The cell numbers were measured by staining DNA of attached cells with Hoechst 33342, and the dose dependent decrease in cell number was expressed relative to untreated cells. As shown in FIG. 13, SNX9 and SNX9-1 displayed pronounced selectivity for partially transformed or fully transformed cell lines, relative to immortal but untransformed fibroblasts. Much lower selectivity for transformed cells was observed with SNX14 and with the selective CDK1 inhibitor, whereas flavopiridol and the selective inhibitors of CDK2 or CDK4 showed no transformed cell selectivity whatsoever. Hence, tumor selectivity of SNX9 class compounds could not be explained by their inhibition of CDK1, CDK2 or CDK4/6. This result places CDK3 (for which no selective chemical inhibitors other than SNX9 class are available) as the top target candidate responsible for tumor selectivity.

### Example 7

### CDK3 Inhibition by RNA Interference Leads to Tumor-Specific Growth-Inhibition

In order to explore SNX9-unrelated specific inhibitors of CDK3, we chose to use recombinant lentiviral vectors that express short hairpin RNA (shRNA) sequences that inhibit CDK3 expression through the RNA interference (RNAi) mechanism. Three lentiviral vectors with different shRNA sequences targeting CDK3 were obtained from Open Biosystems, Huntsville, Ala. These vectors are comprised of pLKO1 backbone (www.openbiosystems.com), which carries a selectable marker for puromycin resistance and expresses shRNA inserts from the human U6 promoter. CDK3-targeting shRNA sequences are listed in Table 3.

TABLE 3
Sequences of CDK3-targeting shRNA.

| | Catalog Number, | |
|---|---|---|
| Name | OpenBiosystems | shRNA targeted sequence |
| 481 | RHS3979-9568852 | GAGAGCAAAGCACTAAGGAAT |
| 482 | RHS3979-9568853 | GCTCTTTCGTATCTTTCGTAT |
| 484 | RHS3979-9568856 | GCAAGTTCTATACCACAGCTG |

In most experiments, we have used a mixture of all three CDK3-targeting lentiviral vectors, to maximize the efficacy of RNAi. As a negative control, we used either an insert-free pLKO1 virus or another insert-free lentiviral vector, LLCEPTu6X, derived from pLL3.7 (Rubinson et al., Nat. Genet. 33, 401-406 (2003) and also carrying a puromycin-resistance marker. To demonstrate that the mixture of three CDK3-targeting lentiviral vectors inhibits CDK3 RNA expression, the virus was prepared by co-transfection of three shRNA vectors with ViraPower lentiviral packaging mix into 293FT cells (Invitrogen), and packaged virus was transduced into HT1080 cells. Cells infected with shRNA-expressing or control virus were selected with 2 μg/ml puromycin for 3 days, and their poly(A)+RNA was purified using Oligotex Direct MRNA kit, Qiagen. As an additional control, RNA was extracted from uninfected and unselected cells. The RNA preparations were then tested by real-time reverse transcription-PCR (Q-PCR) for a decrease in the level of CDK3 mRNA. Q-PCR analysis was carried out using SYBR Green PCR Master Mix (ABI) with ABI 7900HT Q-PCR machine, in triplicate. Serial cDNA dilutions and gel electrophoresis were used for primer validation, and the comparative C_{T} method for relative quantitation of gene expression (Applied Biosystems) was used to determine expression levels. The following PCR primers were used for CDK3: GCCCCCGAGATTCTCTTGG (sense) and GGAAACAGGGCTTTTCGA (antisense); these primers produce a 103 bp PCR product. As a normalization control, β-actin sequences were amplified using the following PCR primers:

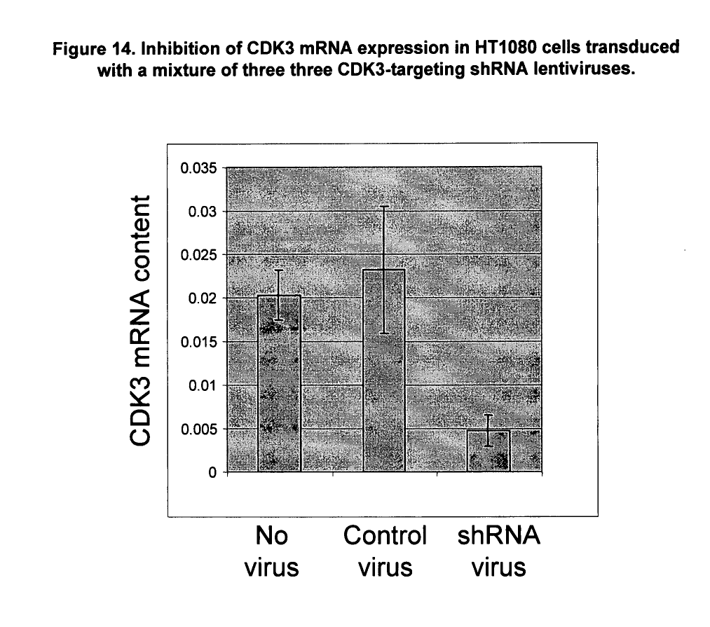

CTTCCTGGGCATGGAGTC (sense) and TGTTGGCGTACAGGTCTTTG (antisense), yielding a 95-bp fragment. FIG. 14 shows the results of Q-PCR analysis of CDK3 mRNA levels (after normalization to β-actin). While these levels were unchanged in cells infected with a control virus, infection with a mixture of three CDK3-targeting shRNA viruses decreased CDK3 mRNA levels approximately 4-fold, indicating efficient RNAi activity.

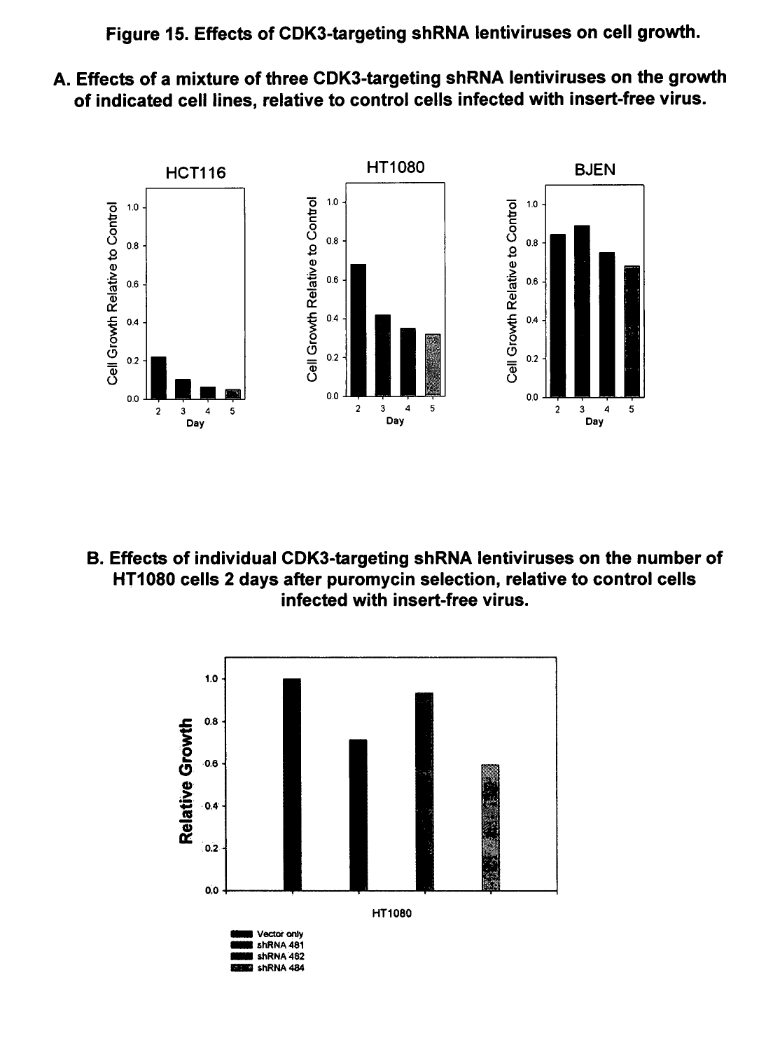

The effects of the CDK3-targeting lentiviral mixture on the growth of normal and tumor cells was determined by using this mixture and the control insert-free virus to infect two tumor cell lines, HT1080 fibrosarcoma and HCT116 colon carcinoma, which we previously found to be highly susceptible to SNX9 class compounds and immortalized normal BJ-EN fibroblasts, which are relatively more resistant to SNX9 class compounds (FIG. 13). The cell growth was monitored after infection and puromycin selection, by plating puromycin-selected cells in 6-well plates and measuring the cell numbers every day using Coulter Z1 counter (in triplicates), for six days. As shown in FIG. 15A, infection with a mixture of three CDK3-targeting lentiviruses drastically inhibited the growth of HCT116 and HT1080 cells, but the inhibitory effect on BJ-EN cells was much weaker. In another experiment, HT1080 cells were infected with three individual lentiviruses carrying shRNA against CDK3, and the cell number was determined two days after puromycin selection. As shown in FIG. 15B, two of three individual lentiviruses (481 and 484) inhibited HT1080 cell growth relative to the control, with the strongest effect obtained with 484 (see Table 3). Hence, RNAi vectors that inhibit CDK3 also inhibit cell growth, and this inhibitory effect is specific for tumor cells, mimicking the effect of SNX9 class compounds (see FIG. 12).

### Example 8

### In Vivo Studies of SNX9 and SNX9-1

Male NCr nude mice, aged approximately 6-8 weeks were used for the study, which was carried out as a service by Taconic Biotechnology, Rensselaer, N.Y. Animals were maintained under virus free barrier conditions with continuous health monitoring. The administration of test materials, all data collection and disposal of study animals was in compliance with all relevant Taconic Biotechnology Standard Operating Procedures as well as The Guide for the Care and Use of Laboratory Animals. Study animals were observed upon arrival and daily throughout the study for overall health, behavior and morbidity. Test animals were subject to body weight measurement to determine dosage volumes of test articles.

The initial part of this analysis was a Range Finder Study, designed to evaluate acute toxicity and to identify the maximum tolerated dose (MTD) for therapeutic study. The Range Finder Study was performed twice. For the first iteration, 30 mice were injected intravenously with SNX9 and SNX9-1 in groups of 3 with phosphate buffered saline (PBS) only, 2.2 mg/kg SNX9, 4.4 mg/kg SNX9 8.8 mg/kg SNX9, 17.6 mg/kg SNX9, 80% PBS: 20% DMSO, 2.2 mg/kg SNX9-1, 4.4 mg/kg SNX9-1 8.8 mg/kg SNX9-1, 17.6 mg/kg SNX9-1. SNX9 was dissolved in PBS and SNX9-1 was dissolved in 80% PBS:20% DMSO. The volume injected per animal was approximately 0.1 ml/injection. One animal died on day one in the 2.2 mg/kg SNX9-1 (lowest dose) group, apparently due to shock. All other animals appeared healthy and thrived for the duration of the 3 day period of observation, and their general health and body weight were assessed. For all the mice, there was no significant weight change (see Table 4). At the end of the 3 day observation period all the mice were euthanized and a terminal blood sample was collected via cardiac puncture. These blood samples were analyzed for complete blood count evaluation. As shown in Table 4, the White Blood Cell count was in each case within the reference range, indicating neither SNX9 nor SNX9-1 had a detectable impact on White Blood Cell count even at the highest dose. A slight elevation of Red Blood Cell count was detected in approximately half of the groups, although this elevation was less than a 7% increase above the upper reference range and was not considered significant. All other blood count categories placed within the reference range.

For the second iteration, 24 mice were injected intravenously with SNX9 and SNX9-1 in groups of 3 with PBS only, 17.6 mg/kg SNX9, 35.2 mg/kg SNX9, 70.4 mg/kg SNX9, 80% PBS:20% DMSO, 17 mg/kg SNX9-1, 35.2 mg/kg SNX9-1, 70.4 mg/kg SNX9-1. One animal died shortly after dosing in the 70.4 mg/kg SNX9 group; this rapid death was likely due to shock and not compound toxicity. All other animals were healthy for the duration of the 3 day period during which the animals were observed and their general health and body weight were assessed. For all the mice, there was no significant weight change (see Table 4). The average beginning weight for the mice in the second iteration was 23.7 g±1.73. After the 3 day observation period all the mice were euthanized and a terminal blood sample was collected via cardiac puncture. These blood samples were analyzed for complete blood count evaluation. As in the first iteration, the White Blood Cell count was in each case within the reference range (Table 4), indicating that neither SNX9 nor SNX9-1 had a detectable impact on White Blood Cell count, even at the highest dose injected. A slight elevation of Red Blood Cell count was detected in approximately 7 out of eight of the groups, although this elevation was less than a 6% increase above the upper reference range and was not considered significant. All other blood count categories placed within the reference range.

TABLE 4

| | Avg. Weight Change | White Blood Cells | Red Blood Cells |
|---|---|---|---|
| Injection-Iteration 1 | Avg. starting weight: 24.9 g +/− 2.00 | Reference Range: 2.6-10.69 × 10³/μl | Reference Range: 6.4-9.4 × 10⁶/μl |
| PBS | 1.8 g +/− 0.36 | 10.2 × 10³/μl +/− 1.40 | 10.05 × 10⁶/μl +/− 0.30 |
| 2.2 mg/kg SNX9 | −0.2 g +/− 1.67 | 3.9 × 10³/μl +/− 1.51 | 8.79 × 10⁶/μl +/− 1.13 |
| 4.4 mg/kg SNX9 | 1.6 g +/− 0.20 | 5.6 × 10³/μl +/− 1.50 | 9.81 × 10⁶/μl +/− 0.11 |
| 8.8 mg/kg SNX9 | −0.2 g +/− 1.77 | 7.3 × 10³/μl +/− 2.60 | 8.42 × 10⁶/μl +/− 0.73 |
| 17.6 mg/kg SNX9 | 0.6 g +/− 0.23 | 8.4 × 10³/μl +/− 0.00 | 9.82 × 10⁶/μl +/− 0.28 |
| PBS:DMSO::80:20 | −0.4 g +/− 0.63 | 5.5 × 10³/μl +/− 0.49 | 9.47 × 10⁶/μl +/− 0.19 |
| 2.2 mg/kg SNX9-1 | 0.8 g +/− 0.25 | 5.2 × 10³/μl +/− 0.28 | 9.84 × 10⁶/μl +/− 0.10 |
| 4.4 mg/kg SNX9-1 | 0.7 g +/− 1.51 | 5.2 × 10³/μl +/− 0.61 | 8.51 × 10⁶/μl +/− 0.89 |
| 8.8 mg/kg SNX9-1 | 1.4 g +/− 0.36 | 6.7 × 10³/μl +/− 1.61 | 9.29 × 10⁶/μl +/− 0.35 |
| 17.6 mg/kg SNX9-1 | 1.1 g +/− 1.06 | 5.0 × 10³/μl +/− 2.95 | 9.07 × 10⁶/μl +/− 0.13 |
| | Avg. Weight Change | White Blood Cells | Red Blood Cells |
| Injection-Iteration 2 | Avg. starting weight: 23.7 g +/− 1.73 | Reference Range: 2.6-10.69 × 10³/μl | Reference Range: 6.4-9.4 × 10⁶/μl |
| PBS | −0.2 g +/− 0.15 | 6.7 × 10³/μl +/− 0.75 | 9.05 × 10⁶/μl +/− 1.70 |
| 17.6 mg/kg SNX9 | −0.4 g +/− 0.12 | 5.2 × 10³/μl +/− 0.96 | 9.92 × 10⁶/μl +/− 0.62 |
| 35.2 mg/kg SNX9 | −0.4 g +/− 0.20 | 7.2 × 10³/μl +/− 3.00 | 9.93 × 10⁶/μl +/− 0.63 |
| 70.4 mg/kg SNX9 | 0.1 g +/− 0.49 | 5.7 × 10³/μl +/− 0.90 | 9.49 × 10⁶/μl +/− 0.50 |
| PBS:DMSO::80:20 | −0.2 g +/− 0.36 | 7.7 × 10³/μl +/− 1.51 | 9.78 × 10⁶/μl +/− 0.32 |
| 17.6 mg/kg SNX9-1 | −0.4 g +/− 0.26 | 6.8 × 10³/μl +/− 1.03 | 9.85 × 10⁶/μl +/− 0.59 |
| 35.2 mg/kg SNX9-1 | 0.0 g +/− 0.44 | 8.0 × 10³/μl +/− 0.76 | 9.73 × 10⁶/μl +/− 0.23 |
| 70.4 mg/kg SNX9-1 | 0.0 g +/− 0.26 | 6.3 × 10³/μl +/− 0.49 | 9.69 × 10⁶/μl +/− 0.11 |

The complete lack of toxicity of SNX9 and SNX9-1 in the Range Finder Study raised a question whether these compounds could be very rapidly lost from the bloodstream. In order to determine the ability of SNX9 to remain in the blood of mice at detectable levels, we analyzed its plasma level one hour after IV injection. Two control mice were uninjected, and two mice were injected IV with 70 mg/kg SNX9 (dissolved in PBS). After one hour, the mice were euthanized and the blood collected by cardiac puncture. After plasma was prepared from the whole blood it was frozen and stored at −70° C. The frozen plasma was thawed and 100 μl was cleared of plasma protein by precipitation with 4 volumes of 100% ethanol at −20° C. for one hour. Under these conditions, SNX9 is recovered quantitatively in the ethanol soluble fraction with a minimum of plasma contamination.

The first assay was HPLC analysis. For this assay, the ethanol soluble fraction was dried under vacuum and re-dissolved in 100 μl 30% acetonitrile 0.1% trifluoroacetic acid (TFA). 4 μl of the ethanol extracted SNX9 was applied to a C18 HPLC column and eluted isocratically using a buffer of 30% acetonitrile 0.1% TFA at a flow rate of 0.3 ml/min. The elution of SNX9 was monitored at its absorption maximum wavelength of 280 nm. Standards were prepared in a like fashion from control plasma spiked with SNX9 at the concentrations of 0 μM, 250 μM, 500 μM, 1 mM and 2 mM. These standards gave a characteristic absorption peak at 280 nm, eluting at approximately 5.5 minutes. The area under each standard peak was proportional to the concentration of SNX9 in the original plasma. In this way the concentration of SNX9 in the plasma of two injected mice was estimated to be close to 250 μM (data not shown).

The second assay was a biological test for cytotoxicity. In this assay, the ethanol soluble fraction was dissolved in DMEM containing 10% Fetal Calf Serum. Standards were prepared from control plasma that was spiked with 1 mM SNX9. The mouse-derived samples were then diluted into DMEM containing 10% Fetal Calf Serum identically to the standard 1 μM sample. These initial dilutions were then serially diluted to 12.5 μM, 6.25 μM, 3.12 μM, 1.56 μM, 0.78 μM, 0.39 μM, 0.195 μM, 0.097 μM, 0.048 μM, 0.024 μM and zero. The sample from the SNX9 injected animal was treated as if the concentration was 1 mM SNX9 in order to normalize for background plasma effects. These control and experimental dilutions were then applied to HT1080 cells seeded at 2000 cells per well of a 96 well tissue culture plate, in triplicate. After 72 hours the growth inhibition was measured by Hoechst staining of the cell lysate in each well of the 96 well tissue culture plate, prepared as previously described. A similar growth inhibition profile was observed in the experimental sample derived from the plasma of mice injected with 70 mg/kg SNX9 and the control plasma with spiked SNX9 at 1 mM (data not shown). From the above assays, we concluded that the active concentration of SNX9 in the plasma of mice injected IV with 70 mg/kg of the compound was between 250 μM and 1 mM, and the lack of toxicity was not due to the immediate loss of the compound from the bloodstream.

With this information, we then carried out the Therapeutic Dose Study of SNX9 and SNX9-1, in nude mice bearing established human colon (HCT116) cancer xenografts. HCT116 was from American Type Culture Collection (ATCC); ATCC information was referenced for the cryopreservation and growing of the cells. The cells were injected at 5×10⁶ subcutaneously in the intra-scapular region. The tumor dimensions were measured using vernier calipers. To approximate the subcutaneous tumor size, the calipers were compressed on the skin slightly but not so tightly as to grip the subcutaneous mass. The maximal dimension of the tumor was recorded as L. The dimension perpendicular to L was recorded as W. The formula: Volume=0.5*L*W² was used. Eight days following tumor cell injection into the intra-scapular region, when all the tumor inoculi became palpable, groups of 10 mice were treated for 21 days with PBS (control), SNX9 or SNX9-1 (each at 70 mg/kg), dissolved as in the Range Finder Study.

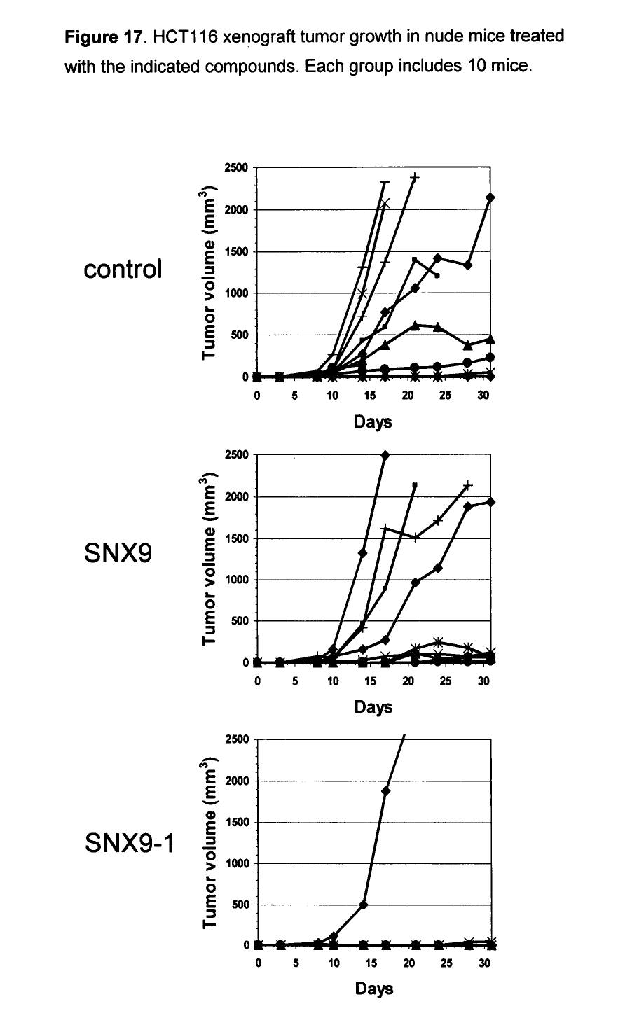

The compounds were injected intra-tumorally three times a week, on alternating days, for a total of 9 injections per animal over three weeks. Mice were sacrificed when tumor size exceeded 2000 mm³. FIG. 17 shows the time course of changes in the tumor size for all the animals. By day 31 (2 days after the end of treatment), 6 of 10 mice in the control group (60%) died or were sacrificed, as compared to 40% of the mice in SNX9-treated group and only 10% of the mice in SNX9-1 treated group. Statistical analysis of differences in the tumor size on day 31 was carried out using paired two-tailed t-test; for this analysis, the tumor volume of all the mice that died or were sacrificed before day 31 was assumed to be 2000 mm³. This analysis showed that the decrease in the tumor size on day 31 was highly significant for the SNX9-1 treated group relative to the control (P<0.01) and did not reach statistical significance for SNX9-treated group (P<0.3). Hence, the Therapeutic Dose Study demonstrated in vivo anti-tumor efficacy of SNX9-class compounds, in particular SNX9-1.

What is claimed is:

1. A method of inhibiting tumor cell growth comprising contacting a population of tumor cells with a compound having the structure I: 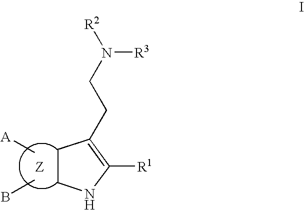
or free acids, salts, or prodrugs thereof;
wherein R¹, R² and R³ are each independently selected from hydrogen, C1-C6 alkyl, cycloalkyl, alkenyl, O-alkyl, dialkylaminoalkyl, aryl and heteroaryl, or R² and R³ together form a ring of 3-6 atoms, which may include one or more heteroatom and/or one or more double bond; A and B are each independently selected from hydrogen, O-alkyl, N-alkyl, and an electron-withdrawing group, provided, however, that at least one of A or B is an electron withdrawing group; and Z is a 4-10 atom ring structure, which contains 0-3 heteroatoms, and one or more double bonds.

2. The method according to claim 1, wherein R¹ is hydrogen or methyl, and R² and R³ are independently selected from hydrogen, methyl and C2-C6 alkyl or alkenyl.

3. The method according to claim 1, wherein one or more electron-withdrawing group is selected from halogen, a nitrogen-containing group, or an oxygen-containing group.

4. The method according to claim 3, wherein one or more halogen is not fluorine.

5. The method according to claim 3, wherein one or more halogen is selected from chlorine and bromine.

6. A method of inhibiting tumor cell growth comprising contacting a population of tumor cells with a compound having the structure II: 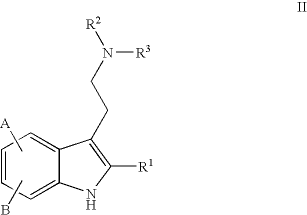
or free acids, salts, or prodrugs thereof;
wherein R¹, R² and R³ are each independently selected from hydrogen, C1-C6 alkyl, cycloalkyl, alkenyl, O-alkyl, dialkylaminoalkyl, aryl and heteroaryl, or R² and R³ together form a ring of 3-6 atoms, which may include one or more heteroatom and/or one or more double bond; and A and B are each independently selected from hydrogen, O-alkyl, N-alkyl and an electron-withdrawing group, provided, however, that at least one of A or B is an electron withdrawing group.

7. The method according to claim 6, wherein R¹ is hydrogen or methyl, and R² and R³ are independently selected from hydrogen, methyl and C2-C6 alkyl or alkenyl.

8. The method according to claim 6, wherein one or more electron-withdrawing group is selected from halogen, a nitrogen-containing group, or an oxygen-containing group.

9. The method according to claim 8, wherein one or more halogen is not fluorine.

10. The method according to claim 8, wherein one or more halogen is selected from chlorine and bromine.

11. A method for inhibiting cancer cell growth comprising contacting a population of cancer cells with a compound selected from: 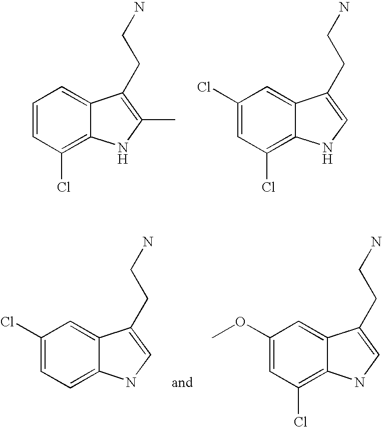
or free acids, salts or prodrugs thereof.

12. A method for treating a patient having a tumor comprising administering to the patient a pharmaceutical formulation comprising a compound having the structure I: 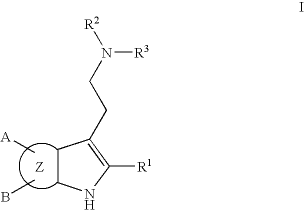
or free acids, salts, or prodrugs thereof and a pharmaceutically acceptable diluent, carrier or excipient;
wherein R¹, R² and R³ are each independently selected from hydrogen, C1-C6 alkyl, cycloalkyl, alkenyl, O-alkyl, dialkylaminoalkyl, aryl and heteroaryl, or R² and R³ together form a ring of 3-6 atoms, which may include one or more heteroatom and/or one or more double bond; A and B are each independently selected from hydrogen, O-alkyl, N-alkyl, and an electron-withdrawing group, provided, however, that at least one of A or B is an electron withdrawing group; and Z is a 4-10 atom ring structure, which contains 0-3 heteroatoms, and one or more double bonds.

13. The method according to claim 12, wherein R¹ is hydrogen or methyl, and R² and R³ are independently selected from hydrogen, methyl and C2-C6 alkyl or alkenyl.

14. The method according to claim 12, wherein one or more electron-withdrawing group is selected from halogen, a nitrogen-containing group, or an oxygen-containing group.

15. The method according to claim 14, wherein one or more halogen is not fluorine.

16. The method according to claim 14, wherein one or more halogen is selected from chlorine and bromine.

17. A method for treating a patient having a tumor comprising administering to the patient a pharmaceutical formulation comprising a compound having the structure II: 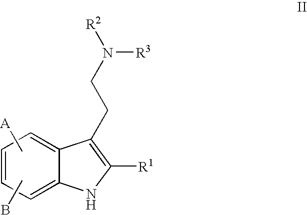
or free acids, salts, or prodrugs thereof and a pharmaceutically acceptable diluent, carrier or excipient;
wherein R¹, R² and R³ are each independently selected from hydrogen, C1-C6 alkyl, cycloalkyl, alkenyl, O-alkyl, dialkylaminoalkyl, aryl and heteroaryl, or R² and R³ together form a ring of 3-6 atoms, which may include one or more heteroatom and/or one or more double bond; and A and B are each independently selected from hydrogen, O-alkyl, N-alkyl and an electron-withdrawing group, provided, however, that at least one of A or B is an electron withdrawing group.

18. The method according to claim 17, wherein R¹ is hydrogen or methyl, and R² and R³ are independently selected from hydrogen, methyl and C2-C6 alkyl or alkenyl.

19. The method according to claim 17, wherein one or more electron-withdrawing group is selected from halogen, a nitrogen-containing group, or an oxygen-containing group.

20. The method according to claim 19, wherein one or more halogen is not fluorine.

22. The method according to claim 20, wherein one or more halogen is selected from chlorine and bromine.

23. A method for treating a patient having a tumor comprising administering to the patient a pharmaceutical formulation comprising a compound having the structure selected from: 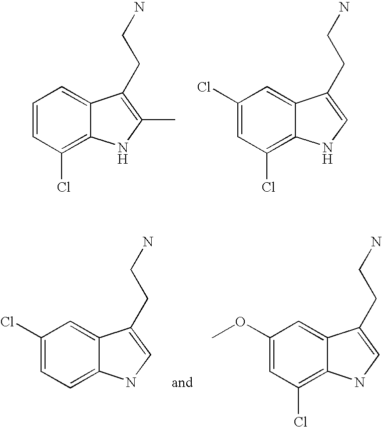
or free acids, salts or prodrugs thereof and a pharmaceutically acceptable diluent, carrier or excipient.

24. A method for preventing or reducing CDKI pathway induced transcription in a cell, comprising contacting the cell with a compound having the structure I: 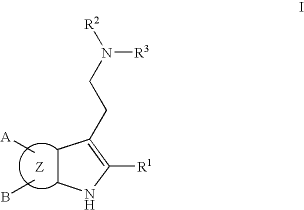
or free acids, salts, or prodrugs thereof and a pharmaceutically acceptable diluent, carrier, or excipient;
wherein R¹, R² and R³ are each independently selected from hydrogen, C1-C6 alkyl, cycloalkyl, alkenyl, O-alkyl, dialkylaminoalkyl, aryl and heteroaryl, or R² and R³ together form a ring of 3-6 atoms, which may include one or more heteroatom and/or one or more double bond; A and B are each independently selected from hydrogen, O-alkyl, N-alkyl, and an electron-withdrawing group, provided, however, that at least one of A or B is an electron withdrawing group; and Z is a 4-10 atom ring structure, which contains 0-3 heteroatoms, and one or more double bonds.

25. The method according to claim 24, wherein R¹ is hydrogen or methyl, and R² and R³ are independently selected from hydrogen, methyl and C2-C6 alkyl or alkenyl.

26. The method according to claim 24, wherein one or more electron-withdrawing group is selected from halogen, a nitrogen-containing group, or an oxygen-containing group.

27. The method according to claim 26, wherein one or more halogen is not fluorine.

28. The method according to claim 26, wherein one or more halogen is selected from chlorine and bromine.

29. A method for selectively inhibiting tumor cell growth comprising selectively inhibiting in a tumor cell cyclin-dependent kinase 3 (CDK3).

30. The method according to claim 29, wherein selectively inhibiting in a tumor cell CDK3 comprises contacting a tumor cell with a small molecule specific inhibitor of CDK3 activity or a dominant negative mutant of CDK3.

31. The method according to claim 30, wherein the small molecule specific inhibitor of CDK3 has structure I or structure II

32. The method according to claim 29 wherein selectively inhibiting in a tumor cell CDK3 comprises contacting a tumor cell with an inhibitor of CDK3 gene expression.

33. The method according to claim 32 wherein the inhibitor of CDK3 gene expression is selected from the group consisting of a short hairpin RNA (shRNA), a small inhibitory RNA (siRNA), an antisense nucleic acid (AS), and a ribozyme.

34. A method for identifying a specific inhibitor of tumor cell growth, the method comprising contacting an in vitro complex of a purified cyclin that interacts with CDK3 and CDK3 under conditions in which the complex of purified cyclin that interacts with CDK3 and CDK3 is capable of exhibiting kinase activity with a candidate inhibitor of such activity, measuring the kinase activity of such complex in the presence or absence of such candidate compound, wherein the candidate compound is regarded as a specific inhibitor of tumor cell growth if the activity of a cyclin/CDK3 complex is lower in the presence of the candidate inhibitor than in the absence of the candidate inhibitor.

35. The method according to claim 34, further comprising using a complex of CDK1, CDK2, CDK4, or CDK6, and cyclins that interact with such CDKs, wherein the candidate inhibitor is regarded as a specific inhibitor of tumor cell growth if the candidate inhibitor inhibits the activity of the cyclin/CDK3 complex to a greater extent than the kinase activity of a complex of CDK1, CDK2, CDK4, or CDK6 with their interacting cyclins.

36. A specific inhibitor of tumor cell growth identified by the method of claim 34 or 35.

37. A method for reducing or preventing CDKI pathway induced transcription in a cell, comprising contacting the cell with a compound having the structure II: 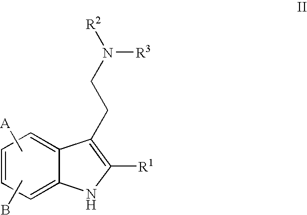
or free acids, salts, or prodrugs thereof and a pharmaceutically acceptable diluent, carrier or excipient;
wherein R¹, R² and R³ are each independently selected from hydrogen, C1-C6 alkyl, cycloalkyl, alkenyl, O-alkyl, dialkylaminoalkyl, aryl and heteroaryl, or R² and R³ together form a ring of 3-6 atoms, which may include one or more heteroatom and/or one or more double bond; and A and B are each independently selected from hydrogen, O-alkyl, N-alkyl and an electron-withdrawing group, provided, however, that at least one of A or B is an electron withdrawing group.

38. The method according to claim 37, wherein R¹ is hydrogen or methyl, and R² and R³ are independently selected from hydrogen, methyl and C2-C6 alkyl or alkenyl.

39. The method according to claim 37, wherein one or more electron-withdrawing group is selected from halogen, a nitrogen-containing group, or an oxygen-containing group.

39. The method according to claim 46, wherein one or more halogen is not fluorine.

40. The method according to claim 39, wherein one or more halogen is selected from chlorine and bromine.


SEQUENCE LISTING (29 sequences)

SEQ ID NO: 1 - DNA, 20 bp, Artificial Sequence
  Description of Artificial Sequence Synthetic primer
cgatcgagcatatgttgctg

SEQ ID NO: 2 - DNA, 20 bp, Artificial Sequence
  Description of Artificial Sequence Synthetic primer
atccgagccgttctctacaa

SEQ ID NO: 3 - DNA, 19 bp, Artificial Sequence
  Description of Artificial Sequence Synthetic primer
ggagtgggtgtgtgacgag

SEQ ID NO: 4 - DNA, 18 bp, Artificial Sequence
  Description of Artificial Sequence Synthetic primer
ggagcctaccctgccact

SEQ ID NO: 5 - DNA, 19 bp, Artificial Sequence
  Description of Artificial Sequence Synthetic primer
cacagcaccaggccataga

SEQ ID NO: 6 - DNA, 19 bp, Artificial Sequence
  Description of Artificial Sequence Synthetic primer
ttcgagttgcgcttcaaac

SEQ ID NO: 7 - DNA, 18 bp, Artificial Sequence
  Description of Artificial Sequence Synthetic primer
gttccttggcgaggcttt

SEQ ID NO: 8 - DNA, 20 bp, Artificial Sequence
  Description of Artificial Sequence Synthetic primer
gcttcctgccagacccttac

SEQ ID NO: 9 - DNA, 20 bp, Artificial Sequence
  Description of Artificial Sequence Synthetic primer
cctaacggtggtggagaacc

SEQ ID NO: 10 - DNA, 20 bp, Artificial Sequence
  Description of Artificial Sequence Synthetic primer
ggaccaaaacctgcattgat

SEQ ID NO: 11 - DNA, 18 bp, Artificial Sequence
  Description of Artificial Sequence Synthetic primer
cttcctgggcatggagtc

SEQ ID NO: 12 - DNA, 18 bp, Artificial Sequence
  Description of Artificial Sequence Synthetic primer
agttcacacgtcccatgt

SEQ ID NO: 13 - DNA, 18 bp, Artificial Sequence
  Description of Artificial Sequence Synthetic primer
ctggtgacgcctcttggt

SEQ ID NO: 14 - DNA, 20 bp, Artificial Sequence
  Description of Artificial Sequence Synthetic primer
ccaggcagttggctctaatc

SEQ ID NO: 15 - DNA, 20 bp, Artificial Sequence
  Description of Artificial Sequence Synthetic primer
ccgtgcccagaattgttatc

SEQ ID NO: 16 - DNA, 18 bp, Artificial Sequence
  Description of Artificial Sequence Synthetic primer
tctgccaggctcgacatc

SEQ ID NO: 17 - DNA, 19 bp, Artificial Sequence
  Description of Artificial Sequence Synthetic primer
tcaggtggctcttcctcct

SEQ ID NO: 18 - DNA, 19 bp, Artificial Sequence
  Description of Artificial Sequence Synthetic primer
ccccgagcatggaagtatt

SEQ ID NO: 19 - DNA, 20 bp, Artificial Sequence
  Description of Artificial Sequence Synthetic primer
ccaattttcaagcacacgaa

SEQ ID NO: 20 - DNA, 20 bp, Artificial Sequence
  Description of Artificial Sequence Synthetic primer
cagccgtcagcttctcctta

SEQ ID NO: 21 - DNA, 19 bp, Artificial Sequence
  Description of Artificial Sequence Synthetic primer
ctggatggtcactggttgg

SEQ ID NO: 22 - DNA, 20 bp, Artificial Sequence
  Description of Artificial Sequence Synthetic primer
tgttggcgtacaggtctttg

SEQ ID NO: 23 - DNA, 21 bp, Artificial Sequence
  Description of Artificial Sequence Synthetic oligonucleotide
gagagcaaagcactaaggaat

SEQ ID NO: 24 - DNA, 21 bp, Artificial Sequence
  Description of Artificial Sequence Synthetic oligonucleotide
gctctttcgtatctttcgtat

SEQ ID NO: 25 - DNA, 21 bp, Artificial Sequence
  Description of Artificial Sequence Synthetic oligonucleotide
gcaagttctataccacagctg

SEQ ID NO: 26 - DNA, 19 bp, Artificial Sequence
  Description of Artificial Sequence Synthetic primer
gcccccgagattctcttgg

SEQ ID NO: 27 - DNA, 18 bp, Artificial Sequence
  Description of Artificial Sequence Synthetic primer
ggaaacagggcttttcga

SEQ ID NO: 28 - DNA, 18 bp, Artificial Sequence
  Description of Artificial Sequence Synthetic primer
cttcctgggcatggagtc

SEQ ID NO: 29 - DNA, 20 bp, Artificial Sequence
  Description of Artificial Sequence Synthetic primer
tgttggcgtacaggtctttg